(19) United States
(12) Patent Application Publication
(10) Pub. No.: US 2010/0222336 A1
(43) Pub. Date: Sep. 2, 2010

(54) SINGLE ENANTIOMER BETA-AGONISTS, METHODS FOR THE PRODUCTION THEREOF AND THE USE THEREOF AS MEDICATION

(75) Inventors: Ingo Konetzki, Warthausen (DE); Peter Sieger, Mittelbiberach (DE); Marco Santagostino, Mittelbiberach (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/376,226

(22) PCT Filed: Aug. 3, 2007
(86) PCT No.: PCT/EP07/58051

§ 371 Date: Apr. 9, 2009

(30) Foreign Application Priority Data
Aug. 7, 2006 (EP) 06118524.5

(51) Int. Cl. A61K 31/538; C07D 413/12; A61P 11/00

(52) U.S. Cl. 514/230.5; 544/105

(57) ABSTRACT

The present invention relates to single enantiomer compounds of formula (1), where the moieties n, A, R¹, R², R³, m, and Y can have the meanings stated in the claims and in the description, methods for the production thereof, and the use thereof as medication, in particular as medication to treat diseases of respiratory tract.

The present invention relates to enantiomerically pure compounds of formula 1

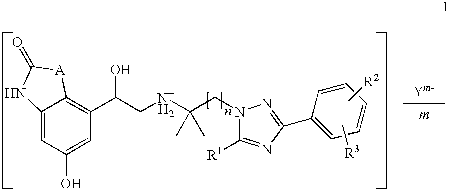

wherein the groups n, A, R¹, R², R³, m and Y may have the meanings given in the claims and specification, processes for preparing them and their use as medicaments, particularly as medicaments for the treatment of respiratory complaints.

### BACKGROUND TO THE INVENTION

Betamimetics (β-adrenergic substances) are known from the prior art. In this respect reference may be made for example to the disclosure of U.S. Pat. No. 4,341,778 which proposes betamimetics for the treatment of a wide range of ailments.

For drug treatment of diseases it is often desirable to prepare medicaments with a longer duration of activity. As a rule, this ensures that the concentration of the active substance in the body needed to achieve the therapeutic effect is maintained for a longer period without the need to re-administer the drug at frequent intervals. Moreover, giving an active substance at longer time intervals contributes to the well-being of the patient to a high degree.

It is particularly desirable to prepare a pharmaceutical composition which can be used therapeutically by administration once a day (single dose). The use of a drug once a day has the advantage that the patient can become accustomed relatively quickly to regularly taking the drug at certain times of the day.

The aim of the present invention is therefore to provide betamimetics which on the one hand provide a therapeutic benefit in the treatment of respiratory complaints and are also characterised by a longer duration of activity and can thus be used to prepare pharmaceutical compositions with a longer duration of activity. A particular aim of the invention is to prepare betamimetics which, by virtue of their long-lasting effect, can be used to prepare a drug for the treatment of asthma for administration once a day. In addition to these aims, a further objective of the invention is to provide such betamimetics which are not only exceptionally potent but are also characterised by a high degree of selectivity with respect to the β₂-adreno-receptor. A further aim of the present invention is to provide betamimetics which by virtue of their physicochemical properties can be used especially for the preparation of pharmaceutical formulations that are particularly suitable for use by inhalation. In particular, the present invention sets out to provide betamimetics which in addition to having the above-mentioned properties are also particularly suitable for the production of inhalable powders and suspension aerosols.

### DETAILED DESCRIPTION OF THE INVENTION

Surprisingly it has been found that the abovementioned problems are solved by compounds of general formula 1. The present invention relates to enantiomerically pure compounds of formula 1

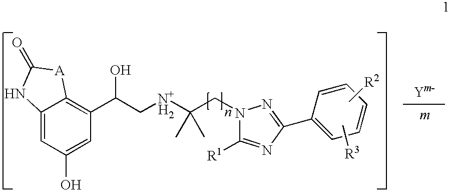

wherein
n denotes 1, 2, 3 or 4;
A denotes a double-bonded group selected from among O, S, CR⁴R⁵, CR⁴R⁵—O, CR⁴R⁵—NR⁶, CH═CH or CH₂—CF₁₂;
R¹ denotes C₁₋₆-alkyl;
R² and R³ which may be identical or different represent H, C₁₋₆-alkyl, C₂₋₆-alkenyl, C₂₋₆-alkynyl, C₃₋₆-cycloalkyl, C₁₋₆-haloalkyl, O—C₁₋₆-haloalkyl, halogen, OH, CN, NO₂, O—C₁₋₆-alkyl, C₂₋₆-alkyl-OH, NH₂, NH—C₁₋₆-alkyl, N(C₁₋₆-alkyl)₂, NHCO—C₁₋₆-alkyl, NHSO₂—C₁₋₆-alkyl, S—C₁₋₆-alkyl, SO—C₁₋₆-alkyl, SO₂—C₁₋₆-alkyl, SO₂NH₂, SO₂NH—C₁₋₆-alkyl, SO₂N(C₁₋₆-alkyl)₂, CONH₂, CONH—C₁₋₆-alkyl, CON(C₁₋₆-alkyl)₂, CO—C₁₋₆-alkyl, COOH or COO—C₁₋₄-alkyl, or
R² and R³ together denote a 2-bonded group selected from O—CR⁴R⁵—O, O—CR⁴R⁵—NR⁶ or CH═CH—CH═CH;
R⁴ denotes H or C₁₋₆-alkyl;
R⁵ denotes H or C₁₋₆-alkyl;
R⁶ denotes H or C₁₋₆-alkyl;
Y^{m-} denotes an anion with m negative charges preferably selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, propanedisulphonate, benzoate and p-toluenesulphonate;
m denotes 1 or 2;
   optionally in the form of the tautomers, mixtures of the tautomers, hydrates or solvates thereof.

The compounds of formula 1 consist of a molecule with a single positive charge and an anion Y^{m-} with a single charge or a corresponding 1/m share of an anion Y^{m-} with m charges. Thus, for example, two molecules of formula

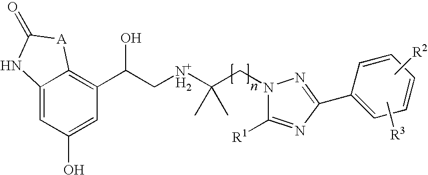

wherein the groups n, A, R¹, R² and R³ may have the above meanings, may be present in a crystalline union with a doubly charged anion Y^{m-} wherein m=2, such as e.g. Ethanedisulphonate or propanedisulphonate.

Preferred are compounds of formula 1 as described above in the form of the enantiomerically pure compounds, while the R-enantiomers of the compounds of formula 1 according to the invention are of exceptional importance. The R-enantiomers of the compounds of formula 1 can be represented by general formula R-1

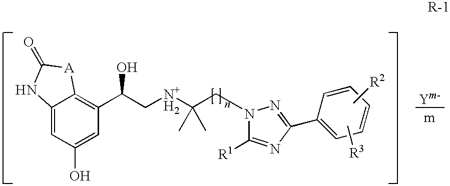

wherein the groups n, A, R¹, R², R³, m and Y may have the meanings given above. The (R)- and (S)-enantiomers may be obtained by common methods known in the art.

Preferred are enantiomerically pure compounds of formula 1, wherein
n denotes 1, 2 or 3, preferably 2;
A denotes a double-bonded group selected from among CR⁴R⁵—O, CH═CH or CH₂—CH₂, preferably CR⁴R⁵—O;
R¹ denotes C₁₋₄-alkyl;
R² and R³ which may be identical or different represent H, C₁₋₄-alkyl, C₂₋₄-alkenyl, C₂₋₄-alkynyl, C₃₋₆-cycloalkyl, C₁₋₄-haloalkyl, O—C₁₋₄-haloalkyl, halogen, OH, CN, NO₂, C₂₋₄-alkyl-OH, O—C₁₋₄-alkyl, COOH or COO—C₁₋₄-alkyl, or
R² and R³ together denote a 2-bonded group selected from O—CR⁴R⁵—O, O—CR⁴R⁵—NR⁶ or CH═CH—CH═CH;
R⁴ denotes H or C₁₋₄-alkyl;
R⁵ denotes H or C₁₋₄-alkyl;
R⁶ denotes H or C₁₋₄-alkyl;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate;
m denotes 1 or 2;
   optionally in the form of the tautomers, mixtures of the tautomers, hydrates or solvates thereof.

Preferred are enantiomerically pure compounds of formula I, wherein
A denotes a double-bonded group selected from among CR⁴R⁵—O, CH═CH or CH₂—CH₂, preferably CR⁴R⁵—O wherein
R⁴ denotes H, methyl, ethyl, preferably H or methyl, particularly preferably H;
R⁵ denotes H, methyl, ethyl, preferably H or methyl, particularly preferably H;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate;
m denotes 1 or 2;
   and wherein n, R¹, R², R³ and R⁶ may each have one of the meanings given hereinbefore or hereinafter, optionally in the form of the tautomers, mixtures of the tautomers, hydrates or solvates thereof.

Preferred are enantiomerically pure compounds of formula 1, wherein R¹ denotes methyl, ethyl or propyl, preferably methyl or ethyl, particularly preferably methyl and wherein n, A, R², R³, R⁴, R⁵, R⁶, m and Y may each have one of the meanings given hereinbefore or hereinafter, optionally in the form of the tautomers, mixtures of the tautomers, hydrates or solvates thereof.

Preferred are enantiomerically pure compounds of formula 1, wherein
R² denotes H, methyl, ethyl, propyl, vinyl, allyl, propargyl, cyclopropyl, cyclopentyl, cyclohexyl, CH₂Cl, CHCl₂, CCl₃, CH₂F, CHF₂, CF₃, CH₂—CH₂Cl, CH₂—CHCl₂, CH₂—CCl₃, CH₂—CH₂F, CH₂—CHF₂, CH₂—CF₃, CH₂—CH₂OH, fluorine, chlorine, bromine, OH, CN, NO₂, methoxy, ethoxy, propoxy, COOH, COO-methyl, COO-ethyl, COO-propyl or COO-butyl;
R³ denotes methyl, ethyl, propyl, vinyl, allyl, propargyl, cyclopropyl, cyclopentyl, cyclohexyl, CH₂Cl, CHCl₂, CCl₃, CH₂F, CHF₂, CF₃, CH₂—CH₂Cl, CH₂—CHCl₂, CH₂—CCl₃, CH₂—CH₂F, CH₂—CHF₂, CH₂—CF₃, CH₂—CH₂OH, fluorine, chlorine, bromine, OH, CN, NO₂, methoxy, ethoxy, propoxy, COOH, COO-methyl, COO-ethyl, COO-propyl or COO-butyl, or
R² and R³ together denote a 2-bonded group selected from O—CR⁴R⁵—O, O—CR⁴R⁵—NR⁶ or CH═CH—CH═CH;
R⁴ denotes H, methyl, ethyl, preferably H or methyl, particularly preferably H;
R⁵ denotes H, methyl, ethyl, preferably H or methyl, particularly preferably H;
R⁶ denotes H, methyl, ethyl, preferably H or methyl, particularly preferably H;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate;
m denotes 1 or 2;
   and wherein n, A and R¹ may each have one of the meanings given hereinbefore or hereinafter, optionally in the form of the tautomers, mixtures of the tautomers, hydrates or solvates thereof.

Preferred are enantiomerically pure compounds of formula I, wherein
R² denotes H, methyl, ethyl, CF₃, CH₂—CF₃, fluorine, chlorine, OH, methoxy, ethoxy, COOH or COO-methyl;
R³ denotes methyl, ethyl, propyl, vinyl, allyl, cyclopropyl, cyclopentyl, cyclohexyl, CH₂F, CHF₂, CF₃, CH₂—CH₂F, CH₂—CHF₂, CH₂—CF₃, CH₂—CH₂OH, fluorine, chlorine, OH, CN, methoxy, ethoxy, COOH, COO-methyl, COO-ethyl or COO-butyl, or
R² and R³ together denote a 2-bonded group selected from O—CH₂—O, O—CMe₂-O or CH═CH—CH═CH;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate;
m denotes 1 or 2;
   and wherein n, A and R¹ may each have one of the meanings given hereinbefore or hereinafter, optionally in the form of the tautomers, mixtures of the tautomers, hydrates or solvates thereof.

Preferred are enantiomerically pure compounds of formula I, wherein
R² denotes H, methyl, ethyl, CF₃, fluorine, chlorine, OH or methoxy;
R³ denotes methyl, ethyl, cyclopropyl, cyclohexyl, CF₃, fluorine, chlorine, OH, CN, methoxy, ethoxy, COOH, COO-methyl, COO-ethyl or COO-butyl, or
R² and R³ together denote a 2-bonded group selected from O—CH₂—O or CH═CH—CH═CH;
Y^{m-} denotes an anion with m negative charges, preferably an anion with m negative charges selected from among chloride, bromide, iodide, sulphate, methanesulphonate, nitrate, maleinate, acetate, benzoate, citrate, malate, lactate, salicylate, trifluoroacetate, fumarate, tartrate, oxalate, succinate and ethanedisulphonate;
m denotes 1 or 2;
   and wherein n, A and R¹ may each have one of the meanings given hereinbefore or hereinafter, optionally in the form of the tautomers, mixtures of the tautomers, hydrates or solvates thereof.

Preferred are enantiomerically pure compounds of formula 1, wherein
R² denotes H, methyl, fluorine, chlorine, OH or methoxy;
R³ methyl, ethyl, CF₃, fluorine, chlorine, OH, methoxy, ethoxy, COOH, COO-methyl or COO-butyl, or
R² and R³ together denote a 2-bonded group selected from O—CH₂—O or CH═CH—CH═CH, preferably O—CH₂—O;
Y^{m-} denotes an anion with m negative charges, preferably an anion with m negative charges selected from among chloride, bromide, iodide, sulphate, methanesulphonate, nitrate, maleinate, acetate, benzoate, citrate, malate, lactate, salicylate, trifluoroacetate, fumarate, tartrate and oxalate;
m denotes 1 or 2;
   and wherein n, A and R¹ may each have one of the meanings given hereinbefore or hereinafter, optionally in the form of the tautomers, mixtures of the tautomers, hydrates or solvates thereof.

Preferred are enantiomerically pure compounds of formula 1, wherein R² denotes hydrogen and wherein n, A, R¹ and R³ in each case may have one of the meanings given above or hereinafter, optionally in the form of the tautomers, mixtures of the tautomers, hydrates or solvates thereof.

Preferred are enantiomerically pure compounds of formula I, wherein n denotes two and wherein A, R¹, R² and R³ may each have one of the meanings given hereinbefore or hereinafter, optionally in the form of the tautomers, mixtures of the tautomers, hydrates or solvates thereof.

Preferred are enantiomerically pure compounds of formula 1, wherein
R³ denotes methyl, ethyl, CF₃, fluorine, chlorine, OH, methoxy, ethoxy, COOH, COO-methyl or COO-butyl, preferably methyl, CF₃, fluorine, chlorine, OH, methoxy, COOH or COO-methyl, particularly preferably methyl, CF₃, fluorine, chlorine, methoxy or COOH;
Y^{m-} denotes an anion with m negative charges, preferably an anion with m negative charges selected from among chloride, bromide, iodide, sulphate, methanesulphonate, maleinate, acetate, benzoate, citrate, malate, lactate, salicylate, fumarate, tartrate and oxalate;
m denotes 1 or 2;
   and wherein n, A, R¹ and R² may each have one of the meanings given hereinbefore or hereinafter, optionally in the form of the tautomers, mixtures of the tautomers, hydrates or solvates thereof.

Of the compounds of formula 1 wherein A denotes CH₂—O, preferred regioisomers are those which are characterised by general formula 1.1.

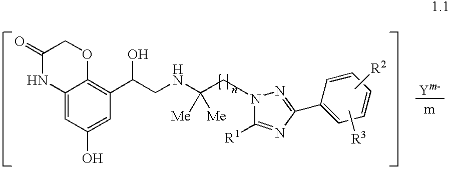

In a preferred aspect the present invention relates to compounds of general formula 1.1 wherein n, R¹, R², R³, m and Y may have the meanings given above. Particularly preferred are the R-enantiomers of the compounds of formula 1.1.

Compounds of formula 1, wherein A denotes CH═CH are characterised by general formula 1.2.

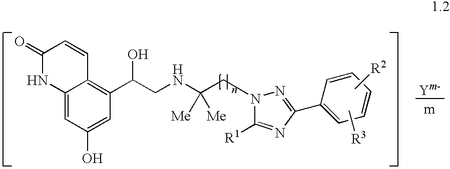

In a preferred aspect the present invention relates to compounds of general formula 1.2 wherein n, R¹, R², R³, m and Y may have the meanings given above. Particularly preferred are the R-enantiomers of the compounds of formula 1.2.

compounds of formula I, wherein A denotes CH₂—CH₂, are characterised by general formula 1.3.

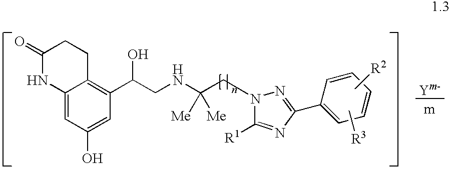

In a preferred aspect the present invention relates to compounds of general formula 1.3 wherein n, R¹, R², R³, m and Y may have the meanings given above. Particularly preferred are the R-enantiomers of the compounds of formula 1.3.

Of the compounds of formula 1, wherein A denotes CR⁴R⁵—O and R⁴ and R⁵ denote methyl, the preferred regioisomers are those which are characterised by general formula 1.4.

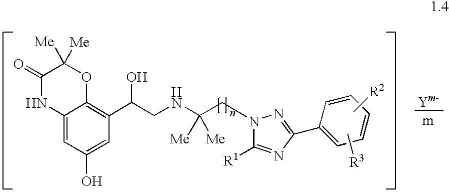

In a preferred aspect the present invention relates to compounds of general formula 1.4 wherein n, R¹, R², R³, m and Y may have the meanings given above. Particularly preferred are the R-enantiomers of the compounds of formula 1.4.

The following enantiomerically pure compounds of formula 1 are preferred,
1.1: 8-(2-{3-[3-(4-fluoro-phenyl)-5-methyl-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.2: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one*H(Y^{m-}/m);
1.3: 8-(2-{1,1-dimethyl-3-[5-methyl-3-(4-trifluoromethyl-phenyl)[1,2,4]triazol-1-yl]-propylamino}-1-hydroxy-ethyl)-6-hydroxy-4H-benzo[1,4]oxazin-3-one*H(Y^{m-}/m);
1.4: 8-(2-{3-[3-(3,5-difluoro-phenyl)-5-methyl-[1,2,4]triazol-1-yl]-1,1-dimethyl-propylamino}-1-hydroxy-ethyl)-6-hydroxy-4H-benzo[1,4]oxazin-3-one*H(Y^{m-}/m);

1.5: 3-(1-{3-[2-hydroxy-2-(6-hydroxy-3-oxo-3,4-dihydro-2H-benzo[1,4]oxazin-8-yl)-ethylamino]-3-methyl-butyl}-5-methyl-1H-[1,2,4]triazol-3-yl-benzoic acid*H(Y^{m-}/m);
1.6: 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.7: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.8: 6-hydroxy-8-(1-hydroxy-2-{3-[3-(4-methoxy-phenyl)-5-methyl-[1,2,4]triazol-1-yl]-1,1-dimethyl-propylamino}-ethyl)-4H-benzo[1,4]oxazin-3-one*H(Y^{m-}/m);
1.9: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one*H(Y^{m-}/m);
1.10: 7-{2-[3-(3-benzo[1,3]dioxol-5-yl-5-methyl-[1,2,4]triazol-1-yl)-1,1-dimethyl-propylamino]-1-hydroxy-ethyl}-5-hydroxy-3H-benzoxazol-2-one*H(Y^{m-}/m) and
1.11: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-2,2-dimethyl-4H-benzo[1,4]oxazin-3-one*H(Y^{m-}/m),
   wherein
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate,
m denotes 1 or 2
   optionally in the form of the tautomers, mixtures of the tautomers, hydrates or solvates thereof.
   Particularly preferred are enantiomerically pure compounds of formula 1,
1.2: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one*H(Y^{m-}/m);
1.3: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one*H(Y^{m-}/m);

1.6: 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.7: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.9: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one*H(Y^{m-}/m);
   wherein
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate,
m denotes 1 or 2
   optionally in the form of the tautomers, mixtures of the tautomers, hydrates or solvates thereof.

Particularly preferred are enantiomerically pure compounds of formula 1, wherein the groups R¹, R² and R³ may have the above meanings and wherein Y^{m-} denotes chloride or bromide, optionally in the form of the tautomers, mixtures of the tautomers, hydrates or solvates thereof.

Particularly preferred are enantiomerically pure compounds of formulae 1.1 to 1.11,

wherein Y′ denotes chloride, bromide, malate (salts of malic acid), maleate or lactate, optionally in the form of the tautomers, mixtures of the tautomers, hydrates or solvates thereof.

Particularly preferred are enantiomerically pure compounds of formula
1.2: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one*H(Y^{m-}/m);
1.3: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one*H(Y^{m-}/m);
1.6: 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.7: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one*H(Yⁿⁱ⁻/m);
1.9: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one*H(Y^{m-}/m);
   wherein Y^{m-} denotes chloride, bromide, malate, maleate or lactate, optionally in the form of the tautomers, mixtures of the tautomers, hydrates or solvates thereof.

Also particularly preferred are the above enantiomerically pure compounds of general formula 1 in crystalline form, optionally in the form of the crystalline tautomers, crystalline hydrates or crystalline solvates thereof. Particularly preferred are the above enantiomerically pure, crystalline compounds of general formula 1 optionally in the form of the crystalline tautomers, crystalline hydrates or crystalline solvates thereof, which are further characterised in that they are crystalline compounds that are present in a single crystal modification.

By the term “single crystal modification” are meant crystalline compounds of formula I that do not constitute a mixture of any existing crystal modifications.

### TERMS AND DEFINITIONS USED

By the term “C₁₋₆-alkyl” (including those which are part of other groups) are meant branched and unbranched alkyl groups with 1 to 6 carbon atoms and by the term “C₁₋₄-alkyl” are meant branched and unbranched alkyl groups with 1 to 4 carbon atoms. Alkyl groups with 1 to 4 carbon atoms are preferred. Examples include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc. may optionally also be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₂₋₆-alkenyl” (including those which are part of other groups) are meant branched and unbranched alkenyl groups with 2 to 6 carbon atoms and by the term “C₂₋₄-alkenyl” are meant branched and unbranched alkenyl groups with 2 to 4 carbon atoms, provided that they have at least one double bond. Alkenyl groups with 2 to 4 carbon atoms are preferred. Examples of these include: ethenyl or vinyl, propenyl, butenyl, pentenyl, or hexenyl. Unless stated otherwise, the definitions propenyl, butenyl, pentenyl and hexenyl include all the possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

By the term “C₂₋₆-alkynyl” (including those which are part of other groups) are meant branched and unbranched alkynyl groups with 2 to 6 carbon atoms and by the term “C₂₋₄-alkynyl” are meant branched and unbranched alkynyl groups with 2 to 4 carbon atoms, provided that they have at least one triple bond. Alkynyl groups with 2 to 4 carbon atoms are preferred. Examples of these include: ethynyl, propynyl, butynyl, pentynyl or hexynyl. Unless stated otherwise, the definitions propynyl, butynyl, pentynyl and hexynyl include all the possible isomeric forms of the groups in question. Thus for example propynyl includes 1-propynyl and 2-propynyl, butynyl includes 1,2- and 3-butynyl, 1-methyl-1-propynyl, 1-methyl-2-propynyl etc.

By the term “C₅₋₆-cycloalkyl” (including those which are part of other groups) are meant cyclic alkyl groups with 5 or 6 carbon atoms. Examples of these include: cyclopentyl or cyclohexyl. Unless otherwise stated, the cyclic alkyl groups may be substituted by one or more groups selected from among methyl, ethyl, iso-propyl, tert-butyl, hydroxy, fluorine, chlorine, bromine and iodine.

By the term “C₁₋₆-haloalkyl” (including those which are part of other groups) are meant branched and unbranched alkyl groups with 1 to 6 carbon atoms, which are substituted by one or more halogen atoms. By the term “C₁₋₄-alkyl” are meant branched and unbranched alkyl groups with 1 to 4 carbon atoms, which are substituted by one or more halogen atoms. Alkyl groups with 1 to 4 carbon atoms are preferred. Preferred halogen atoms are fluorine, chlorine, particularly preferably fluorine. Examples of these include: CF₃, CHF₂, CH₂F, CH₂CF₃.

Halogen within the scope of the present invention represents fluorine, chlorine, bromine or iodine. Unless stated to the contrary, fluorine, chlorine and bromine are regarded as preferred halogens.

The term enantiomerically pure describes, within the scope of the present invention, compounds of formula 1 which are present in an enantiomeric purity of at least 85% ee, preferably at least 90% ee, particularly preferably >95% ee. The term ee (enantiomeric excess) is known in the art and describes the optical purity of chiral compounds.

### Indications

The compounds of formula 1 according to the invention are characterised by their versatility of use in the therapeutic field. Particular mention should be made according to the invention of the possible applications for which the compounds according to the invention of formula 1 are preferably used on account of their pharmaceutical activity as betamimetics.

In another aspect the present invention correspondingly relates to the above-mentioned enantiomerically pure compounds of formula 1 as pharmaceutical compositions. The present invention further relates to the use of the above-mentioned compounds of general formula 1 for preparing a pharmaceutical composition for the treatment of respiratory complaints. The present invention preferably relates to the use of the above-mentioned compounds of general formula 1 for preparing a pharmaceutical composition for the treatment of respiratory complaints, which are selected from among obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the compounds of general formula 1 are used to prepare a pharmaceutical composition for the treatment of obstructive pulmonary diseases selected from among COPD (chronic obstructive pulmonary disease), bronchial asthma, paediatric asthma, severe asthma, acute asthma attacks and chronic bronchitis, while it is particularly preferable according to the invention to use them for preparing a pharmaceutical composition for the treatment of bronchial asthma.

Preferably also, the compounds of formula 1 are used to prepare a pharmaceutical composition for the treatment of pulmonary emphysema which has its origins in COPD (chronic obstructive pulmonary disease) or α1-proteinase inhibitor deficiency.

Preferably also, the compounds of formula 1 are used to prepare a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

Preferably also, the compounds of formula 1 are used to prepare a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

Preferably also, the compounds of general formula 1 are used to prepare a pharmaceutical composition for the treatment of cystic fibrosis or mucoviscidosis.

Preferably also, the compounds of general formula 1 are used to prepare a pharmaceutical composition for the treatment of bronchitis, such as for example bronchitis caused by bacterial or viral infection, allergic bronchitis and toxic bronchitis.

Preferably also, the compounds of general formula 1 are used to prepare a pharmaceutical composition for the treatment of bronchiectasis.

Preferably also, the compounds of general formula 1 are used to prepare a pharmaceutical composition for the treatment of ARDS (adult respiratory distress syndrome).

Preferably also, the compounds of general formula 1 are used to prepare a pharmaceutical composition for the treatment of pulmonary oedema, for example toxic pulmonary oedema after aspiration or inhalation of toxic substances and foreign substances.

Particularly preferably, the present invention relates to the use of the compounds of formula 1 for preparing a pharmaceutical composition for the treatment of asthma or COPD. Also of particular importance is the above-mentioned use of compounds of formula 1 for preparing a pharmaceutical composition for once-a-day treatment of inflammatory and obstructive respiratory complaints, particularly for the once-a-day treatment of asthma or COPD.

The present invention also relates to a process for the treatment of the above-mentioned diseases, characterised in that one or more of the above-mentioned compounds of general formula 1 are administered in therapeutically effective amounts. The present invention further relates to processes for the treatment of asthma or COPD, characterised in that one or more of the above-mentioned compounds of general formula 1 are administered once a day in therapeutically effective amounts.

### EXAMPLES

### Synthesis of the Intermediate Products

### Intermediate product 1: 1,1-dimethyl-3-(5-methyl-3-p-tolyl-[1,2,4]triazol-1-yl)-propylamine

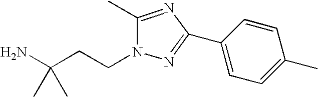

a) 4-methyl-benzoic acid-(1-imino-ethyl)-hydrazide: 1.65 g (72 mmol) sodium are dissolved in 80 mL ethanol. 8.89 g (72 mmol) ethylacetimidate hydrochloride in 160 mL ethanol are added at ambient temperature and the sodium chloride precipitated is filtered off. The filtrate is combined with 6.00 g (40 mmol) 4-methyl-benzoic acid hydrazide and stirred overnight. The reaction mixture is evaporated down and cooled. The solid precipitated is filtered off and washed with cold ethanol and diethyl ether (5.7 g). Another 1.2 g solid are obtained from the filtrate after distillation of the solvents and recrystallisation from ethanol. Yield: 6.93 g (91%); mass spectroscopy [M+H]⁺=192.

b) 5-methyl-3-p-tolyl-[1,2,4]triazole: 7.58 g (40 mmol) 4-methyl-benzoic acid-(1-imino-ethyl)-hydrazide are heated to 180° C. with stirring for 30 minutes. After cooling the solid is dissolved in chloroform. The precipitate formed on cooling is suction filtered and recrystallised from chloroform. Yield: 4.82 g (70%); mass spectroscopy [M+H]⁺=174.

c) tert-butyl[1,1-dimethyl-3-(5-methyl-3-p-tolyl-[1,2,4]triazol-1-yl)-propyl]-carbamate: 1.35 g (34 mmol, 60%) sodium hydride are added at 0° C. to a solution of 4.87 g (28 mmol) 5-methyl-3-p-tolyl-[1,2,4]triazole in 40 mL DMPU. The reaction mixture is heated to ambient temperature and then stirred for one hour. 9.35 g (42 mmol) tert-butyl (3-chloro-1,1-dimethyl-propyl)-carbamate and 1.87 g (5 mmol) tetrabutylammonium iodide are added and the mixture is stirred overnight at ambient temperature and then for another 2 hours at 80° C. Water and ethyl acetate are added, the aqueous phase is separated off and extracted with ethyl acetate. The combined organic phases are washed with water and sodium chloride solution, dried on sodium sulphate and evaporated down. The residue is purified by column chromatography (silica gel; petroleum ether/ethyl acetate=1:1). Oil.

Yield: 2.97 g (30%); mass spectroscopy [M+H]⁺=359.

d) 1,1-dimethyl-3-(5-methyl-3-p-tolyl-[1,2,4]triazol-1-yl)-propylamine. A total of 11 mL of trifluoroacetic acid are added dropwise to a solution of 2.97 g (8.3 mmol) tert-butyl[1,1-dimethyl-3-(5-methyl-3-p-tolyl-[1,2,4]triazol-1-yl)-propyl]-carbamate in 80 mL dichloromethane and the mixture is stirred overnight at ambient temperature. The solvent is distilled off and the residue is combined with diethyl ether and stirred. The solid precipitated is filtered off and washed.

Yield: 2.11 g (68%, trifluoroacetate); mass spectroscopy [M+H]⁺=259.

### Intermediate product 2: 3-[3-(4-fluoro-phenyl)-5-methyl-[1,2,4]triazol-1-yl]-1,1-dimethyl-propylamine

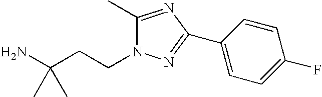

a) 4-fluoro-benzoic acid-(1-imino-ethyl)-hydrazide: prepared from 7.2 g (58 mmol) ethylacetimidate hydrochloride and 5.00 g (32 mmol) 4-fluoro-benzoic acid hydrazide analogously to the method described for intermediate product 1a).

Yield: 5.78 g (91%); mass spectroscopy [M+H]⁺=196.

b) 3-(4-fluoro-phenyl)-5-methyl-[1,2,4]triazole: The preparation is carried out analogously to the method used for intermediate product 1b) from 5.77 g (30 mmol) 4-fluoro-benzoic acid-(1-imino-ethyl)-hydrazide. Yield: 4.11 g (78%); mass spectroscopy [M+H]⁺=178.

c) tert-butyl {3-[3-(4-fluoro-phenyl)-5-methyl-[1,2,4]triazol-1-yl]-1,1-dimethyl-propyl}-carbamate: 5.88 g (33 mmol) 3-(4-fluoro-phenyl)-5-methyl-[1,2,4]triazole are dissolved in 40 mL DMPU and reacted in the manner described for intermediate product 1c) with 11.04 g (50 mmol) tert-butyl (3-chloro-1,1-dimethyl-propyl)-carbamate, 1.59 g (40 mmol, 60%) sodium hydride and 2.21 g (6 mmol) tetrabutylammonium iodide.

Yield: 4.22 g (35%); mass spectroscopy [M+H]⁺=363.

d) 3-[3-(4-fluoro-phenyl)-5-methyl-[1,2,4]triazol-1-yl]-1,1-dimethyl-propylamine: obtained by reacting 4.22 g (116 mmol) tert-butyl {3-[3-(4-fluoro-phenyl)-5-methyl-[1,2,4]triazol-1-yl]-1,1-dimethyl-propyl}-carbamate in 100 mL dichloromethane and 15 mL trifluoroacetic acid. Yield: 4.43 g (trifluoroacetate); mass spectroscopy [M+H]⁺=263.

### Intermediate product 3: 3-[3-(3,5-difluoro-phenyl)-5-methyl-[1,2,4]triazol-1-yl]-1,1-dimethyl-propylamine

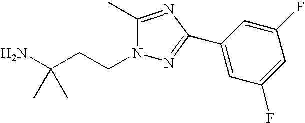

a) 3,5-difluoro-benzoic acid-(1-imino-ethyl)-hydrazide: obtained from 4.91 g (40 mmol) ethylacetimidate hydrochloride and 3.80 g (22 mmol) 3,5-difluoro-benzoic acid hydrazide analogously to the method described for intermediate product 1a).

Yield: 4.49 g (95%); mass spectroscopy [M+H]⁺=214.

b) 3-(3,5-difluoro-phenyl)-5-methyl-[1,2,4]triazole: prepared from 4.61 g (22 mmol) 3,5-difluoro-benzoic acid-(1-imino-ethyl)-hydrazide.

Yield: 3.81 g (91%); mass spectroscopy [M+H]⁺=196.

c) tert-butyl {3-[3-(3,5-difluoro-phenyl)-5-methyl-[1,2,4]triazol-1-yl]-1,1-dimethyl-propyl}-carbamate: 3.74 g (19 mmol) 3-(3,5-difluoro-phenyl)-5-methyl-[1,2,4]triazole in 25 mL DMPU are reacted with 0.92 g (23 mmol, 60%) sodium hydride, 6.37 g (29 mmol) tert-butyl (3-chloro-1,1-dimethyl-propyl)-carbamate and 1.27 g (3 5 mmol) tetrabutylammonium iodide analogously to intermediate product 1c).

Yield: 2.62 g (36%); mass spectroscopy [M+H]⁺=381.

d) 3-[3-(3,5-difluoro-phenyl)-5-methyl-[1,2,4]triazol-1-yl]-1,1-dimethyl-propylamine: 2.62 g (6.9 mmol) tert-butyl{3-[3-(3,5-difluoro-phenyl)-5-methyl-[1,2,4]triazol-1-yl]-1,1-dimethyl-propyl}-carbamate in 65 mL dichloromethane are reacted with 9 mL trifluoroacetic acid in the manner described for intermediate product 1d).

Yield: 2.11 g (trifluoroacetate); mass spectroscopy [M+H]⁺=281.

### Intermediate product 4: 3-[5-ethyl-3-(4-methoxy-phenyl)-[1,2,4]triazol-1-yl]-1,1-dimethyl-propylamine

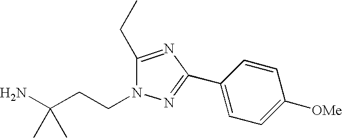

a) 4-methoxy-benzoic acid-(1-imino-propyl)-hydrazide: prepared from 4.90 g (45 mmol) propioamidine hydrochloride and 5.00 g (30 mmol) 4-methoxy-benzoic acid hydrazide analogously to the method described for intermediate product 1a). After the ethanol has been distilled off 10.0 g crude product are obtained, which are reacted without further purification.

b) 5-ethyl-3-(4-methoxy-phenyl)-[1,2,4]triazole: 9.99 g (60%, approx. 28 mmol) 4-methoxy-benzoic acid-(1-imino-propyl)-hydrazide are heated to 150° C. for two hours. After cooling the melt is purified by chromatography on a silica gel column (petroleum ether/ethyl acetate=3:7).

Yield: 4.56 g (75% over two stages); mass spectroscopy [M+H]⁺=204.

c) tert-butyl{3-[5-ethyl-3-(4-methoxy-phenyl)-[1,2,4]triazol-1-yl]-1,1-dimethyl-propyl-carbamate: 4.30 g (21.2 mmol) 5-ethyl-3-(4-methoxy-phenyl)-[1,2,4]triazole are dissolved in 30 mL DMPU and cooled to 0° C. 1.02 g (24 mmol, 60%) sodium hydride are then added batchwise under a protective gas atmosphere and the reaction mixture is slowly heated to ambient temperature and then stirred for one hour. 6.10 g (27 5 mmol) tert-butyl (3-chloro-1,1-dimethyl-propyl)-carbamate and 1.41 g (3 8 mmol) tetrabutylammonium iodide are added. The mixture is stirred overnight and then the reaction is ended by the addition of water and ethyl acetate. The aqueous phase is separated off and extracted with ethyl acetate. The combined organic phases are washed with sodium chloride solution, dried on sodium sulphate and evaporated down. The residue is purified by chromatography on a silica gel column (petroleum ether/ethyl acetate=3:7).

Yield: 6.82 g (83%); mass spectroscopy [M+H]⁺=389.

d) 3-[5-ethyl-3-(4-methoxy-phenyl)-[1,2,4]triazol-1-yl]-1,1-dimethyl-propylamine. A total of 20 mL trifluoroacetic acid are added dropwise to a solution of 6.81 g (17 5 mmol) tert-butyl{3-[5-ethyl-3-(4-methoxy-phenyl)-[1,2,4]triazol-1-yl]-1,1-dimethyl-propyl-carbamate in 150 mL dichloromethane. After three hours stirring at ambient temperature the solution is evaporated down and the residue is combined with diethyl ether. The solid is filtered off, washed with diethyl ether and dried.

Yield: 7.86 g (trifluoroacetate); mass spectroscopy [M+H]⁺=289.

### Intermediate product 5: methyl 3-[1-(3-amino-3-methyl-butyl)-5-methyl-1H-[1,2,4]triazol-3-yl]-benzoate

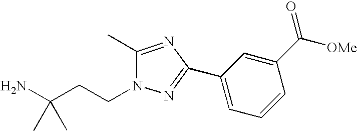

a) methyl 3-[N′-benzyloxycarbonyl-hydrazinocarbonyl)-benzoate: 10.80 g (54 4 mmol) methyl 3-chlorocarbonyl-benzoate in 100 mL diethyl ether are added dropwise to a solution of 9.04 g (54 4 mmol) benzyl hydrazinecarboxylate in 100 mL diethyl ether, 100 mL dichloromethane and 4.83 mL pyridine while cooling with an ice bath. The reaction mixture is stirred overnight at ambient temperature and then mixed with water. The solid precipitated is filtered off and washed with diethyl ether.

Yield: 14.1 g (79%); mass spectroscopy [M−H]⁺=327.

b) methyl 3-hydrazinocarbonyl-benzoate: 14.6 g (44.5 mmol) methyl 3-[N′-benzyloxycarbonyl-hydrazinocarbonyl)-benzoate are dissolved in 75 mL methanol and hydrogenated in the presence of palladium on charcoal (10%) at ambient temperature and 3 bar hydrogen pressure. The catalyst is filtered off and the filtrate is freed from solvent.

Yield: 7.98 g (92%); mass spectroscopy [M+H]⁺=195.

c) methyl 3-[N′-(1-imino-ethyl)-hydrazinocarbonyl]-benzoate: prepared analogously to the method described for intermediate product 1a) from methyl 3-hydrazinocarbonyl-benzoate and ethylacetimidate hydrochloride.

Yield: 8.60 g (90%); mass spectroscopy [M+H]⁺=236.

d) methyl 3-(5-methyl-1H-[1.24]triazol-3-yl)-benzoate: 8.10 g (34.4 mmol) methyl 3-[N′-(1-imino-ethyl)-hydrazinocarbonyl]-benzoate are heated to 180° C. for 30 minutes. 80 mL chloroform are added to the solid obtained after cooling. The suspension is filtered and the product is dried. Yield: 4.03 g (55%); mass spectroscopy [M+H]⁺=218.

e) methyl 3-[1-(3-tert-butoxycarbonylamino-3-methyl-butyl)-5-methyl-1H-[1,2,4]triazol-3-yl-benzoate: 6.00 g (27.6 mmol) methyl 3-(5-methyl-1H-[1.24]triazol-3-yl)-benzoate and 9.19 g (41.4 mmol) tert-butyl (3-chloro-1,1-dimethyl-propyl)-carbamate are reacted and worked up in the manner described for intermediate product 1c).

Yield: 5.96 g (54%); mass spectroscopy [M+H]⁺=403.

f) methyl 3-[1-(3-amino-3-methyl-butyl)-5-methyl-1H-[1,2,4]triazol-3-yl]-benzoate: obtained from methyl 3-[1-(3-tert-butoxycarbonylamino-3-methyl-butyl)-5-methyl-1H-[1,2,4]triazol-3-yl-benzoate analogously to the method described for intermediate product 1d). Yield: 5.36 g (68%, ditrifluoroacetate); mass spectroscopy [M+H]⁺=303.

### Intermediate product 6: methyl 3-[1-(3-amino-3-methyl-butyl)-5-methyl-1H-[1,2,4]triazol-3-yl]-benzoate

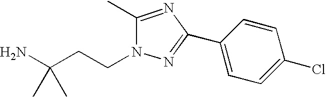

a) 4-chloro-benzoic acid N′-(1-imino-ethyl)-hydrazide: 1.09 g (20 mmol) sodium methoxide in 20 mL ethanol are added to a solution of 1.91 g (20 mmol) acetamidine hydrochloride in 30 mL ethanol. The mixture is stirred for 30 minutes at ambient temperature and then filtered. The filtrate is combined with 2.3 g (13.5 mmol) 4-chlorobenzoic acid hydrazide, stirred overnight at ambient temperature, cooled with an ice bath and then filtered. The precipitate is washed with cold ethanol and dried.

Yield: 1.45 g (51%); mass spectroscopy [M+H]⁺=212/214.

b) 3-(4-chloro-phenyl)-5-methyl-1H-[1.24]triazole: 6.10 g (28.8 mmol) 4-chloro-benzoic acid N′-(1-imino-ethyl)-hydrazide are heated to 180° C. for 30 minutes. After cooling 2.3 g product are obtained from the residue by recrystallisation in chloroform. Evaporation of the mother liquor and subsequent purification of the residue by chromatography (silica gel, petroleum ether/ethyl acetate=1:6) yield an additional 1.22 g product.

Yield: 3.51 g (63%); mass spectroscopy [M+H]⁺=194/196.

c) tert-butyl {3-[3-(4-chloro-phenyl)-5-methyl-[1,2,4]triazol-1-yl]-1,1-dimethyl-propyl}-carbamate: 3.48 g (18.0 mmol) 3-(4-chloro-phenyl)-5-methyl-1H-[1.24]triazole and 5.98 g (27.0 mmol) tert-butyl (3-chloro-1,1-dimethyl-propyl)-carbamate are reacted and worked up in the manner described for intermediate product 1c).

Yield: 3.89 g (57%); mass spectroscopy [M+H]⁺=379/381.

d) methyl 3-[1-(3-amino-3-methyl-butyl)-5-methyl-1H-[1,2,4]triazol-3-yl]-benzoate: obtained from tert-butyl{3-[3-(4-chloro-phenyl)-5-methyl-[1,2,4]triazol-1-yl]-1,1-dimethyl-propyl}-carbamate using the method described for intermediate product 1d).

Yield: 3.65 g (trifluoroacetate); mass spectroscopy [M+H]⁺=279/281.

### Intermediate product 7: 1,1-dimethyl-3-[5-methyl-3-(4-trifluoromethyl-phenyl)-[1,2,4]triazol-1-yl]-propylamine

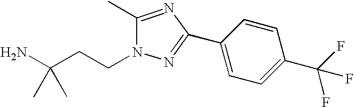

a) 4-trifluoromethyl-benzoic acid N′-(1-imino-ethyl)-hydrazide: 4.78 g (23.4 mmol) 4-(trifluoromethyl)benzoic acid hydrazide and 5.21 g (42.1 mmol) ethylacetimidate hydrochloride are reacted in the manner described for intermediate product 1a). Yield: 6.02 g; mass spectroscopy [M+H]⁺=246.

b) 5-methyl-3-(4-trifluoromethyl-phenyl)-1H-[1,2,4]triazole: prepared from 6.02 g (24.6 mmol) 4-trifluoromethyl-benzoic acid N′-(1-imino-ethyl)-hydrazide analogously to the method described for intermediate product 1b).

Yield: 4.76 g (85%); mass spectroscopy [M+H]⁺=228.

c) tert-butyl{1,1-dimethyl-3-[5-methyl-3-(4-trifluoromethyl-phenyl)-[1,2,4]triazol-1-yl]-propyl}-carbamate: The target compound is obtained analogously to the method described for intermediate product 1c) from 4.90 g (21.6 mmol) 5-methyl-3-(4-trifluoromethyl-phenyl)-1H-[1,2,4]triazole and 7.17 g (32.4 mmol) tert-butyl (3-chloro-1,1-dimethyl-propyl)-carbamate. Yield: 5.06 g (57%); mass spectroscopy [M+H]⁺=413.

d) 1,1-dimethyl-3-[5-methyl-3-(4-trifluoromethyl-phenyl)-[1,2,4]triazol-1-yl]-propylamine. Prepared by the method described for intermediate product 1d) from tert-butyl{1,1-dimethyl-3-[5-methyl-3-(4-trifluoromethyl-phenyl)-[1,2,4]triazol-1-yl]-propyl}-carbamate. Yield: 4.72 g (trifluoroacetate); mass spectroscopy [M+H]⁺=313.

### Intermediate product 8: 3-(3-benzo[1,3]dioxol-5-yl-5-methyl-[1,2,4]triazol-1-yl)-1,1-dimethyl-propylamine

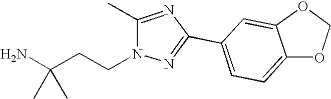

This is prepared analogously to the syntheses described hereinbefore. Mass spectroscopy [M+H]⁺=289.

### Intermediate product 9: 3-[3-(4-methoxy-phenyl)-5-methyl-[1,2,4]triazol-1-yl]-1,1-dimethyl-propylamine

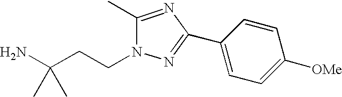

a) 4-methoxy-benzoic acid N′-(1-imino-ethyl)-hydrazide: 4.6 g (0.20 mol) sodium in 200 mL ethanol are combined at ambient temperature with a solution of 25 g (0.20 mol) ethylacetimidate hydrochloride in 200 mL ethanol. The sodium chloride precipitated is suction filtered and 33.2 g (0.20 mol) 4-methoxybenzoic acid hydrazide are added to the filtrate. The reaction mixture is stirred overnight at ambient temperature and then cooled. The precipitate formed is separated off and washed with ethanol and diethyl ether.

Yield: 33.6 g (81%); melting range=179-181° C.

b) 3-(4-methoxy-phenyl)-5-methyl-1H-[1,2,4]triazole: 33.6 g (162 mmol) 4-methoxy-benzoic acid N′-(1-imino-ethyl)-hydrazide are heated to 180° C. for 30 minutes. After cooling the residue is dissolved in 250 mL chloroform and repeatedly extracted with aqueous sodium hydroxide solution. The aqueous phases are combined, washed with chloroform, filtered and adjusted to an acid pH by the addition of glacial acetic acid. The solid precipitated is suction filtered, washed with water and dissolved by heating in chloroform. The solvent is evaporated down and the residue is filtered. The solid is washed with chloroform and diethyl ether. Yield: 23.1 g (75%); melting range=169-171° C.

c) 3-[3-(4-methoxy-phenyl)-5-methyl-[1,2,4]triazol-1-yl]-1,1-dimethyl-propylamine. The target compound is obtained from the reaction of 21.4 g (113 mmol) 3-(4-methoxy-phenyl)-5-methyl-1H-[1,2,4]triazole and 25 g (119 mmol) (3-chloro-1,1-dimethyl-propyl)-[1-phenyl-methylidene]-amine The product is dissolved in 100 mL acetone and acidified with 8.5 mL of 32% aqueous hydrochloric acid and cooled. The hydrochloride precipitated is suction filtered and washed with acetone and diethyl ether.

Yield: 20.3 g; melting range=190-194° C.

### Synthesis of the Salt Precursors

General Method 1: 1 mmol 6-benzyloxy-8-(2-ethoxy-2-hydroxy-acetyl)-4H-benzo[1,4]oxazin-3-one and 1 mmol amine are stirred for 15 minutes in 5 mL tetrahydrofuran at 60° C. The mixture is cooled to 0° C. and under an argon atmosphere 1.5 mL of a 2 molar solution of lithium borohydride in tetrahydrofuran is added dropwise. The mixture is stirred for 15 min at 0° C., combined with 10 mL dichloromethane and 3 mL water, stirred for another hour at ambient temperature and then filtered through kieselguhr, while eluting with dichloromethane. The eluate is freed from the solvent and the residue is purified by chromatography, if necessary. The benzylether thus obtained is dissolved in methanol and hydrogenated with palladium on charcoal (10%) as catalyst at 2.5 bar and ambient temperature. Then the catalyst is separated off and the crude product is purified by chromatography (reverse phase, acetonitrile/water gradient with 0.1% trifluoroacetic acid).

General Method 2: 1 mmol 6-benzyloxy-8-(2-ethoxy-2-hydroxy-acetyl)-4H-benzo[1,4]oxazin-3-one and 1 mmol amine are suspended in 5 mL ethanol and heated to 70° C. The resulting solution is stirred for one hour at 70° C. and then cooled to ambient temperature. After the addition of 113 mg (3 mmol) sodium borohydride the mixture is stirred for 3 hours at ambient temperature, combined with 0.7 mL saturated potassium carbonate solution and stirred for another 30 minutes. It is filtered through aluminium oxide (basic), repeatedly washed with dichloromethane/methanol=15:1, evaporated down and chromatographed (silica gel; dichloromethane with 0-10% methanol:ammonia=9:1). The benzylether thus obtained is dissolved in 10 mL methanol and hydrogenated with palladium on charcoal as catalyst at 1 bar hydrogen pressure. Then the catalyst is filtered off and the filtrate is evaporated down.

### Salt precursor 1: 8-(2-{3-[3-(4-fluoro-phenyl)-5-methyl-[1,2,4]triazol-1-yl]-1,1-dimethyl-propylamino}-1-hydroxy-ethyl)-6-hydroxy-4H-benzo[1,4]oxazin-3-one

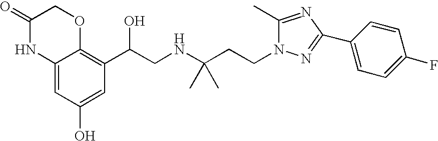

Obtained by reacting 6-benzyloxy-8-(2-ethoxy-2-hydroxy-acetyl)-4H-benzo[1,4]oxazin-3-one and 3-[3-(4-fluoro-phenyl)-5-methyl-[1,2,4]triazol-1-yl]-1,1-dimethyl-propylamine according to General Method 1. Final purification is carried out by chromatography (reverse phase, acetonitrile/water gradient with 0.1% trifluoroacetic acid).

Yield: 134 mg (29%, trifluoroacetate); mass spectroscopy [M+H]⁺=470.

### Salt precursor 2: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one

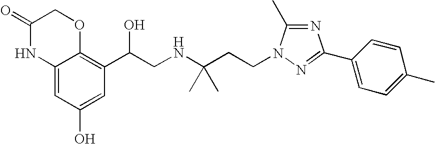

Prepared from 6-benzyloxy-8-(2-ethoxy-2-hydroxy-acetyl)-4H-benzo[1,4]oxazin-3-one and 1,1-dimethyl-3-(5-methyl-3-p-tolyl-[1,2,4]triazol-1-yl]-propylamine according to General Method 1.

Yield: 283 mg (49%, trifluoroacetate); mass spectroscopy [M+H]⁺=466.

### Salt precursor 3: 8-(2-{1,1-dimethyl-3-[5-methyl-3-(4-trifluoromethyl-phenyl)-[1,2,4]triazol-1-yl]propylamino}-1-hydroxy-ethyl)-6-hydroxy-4H-benzo[1,4]oxazin-3-one

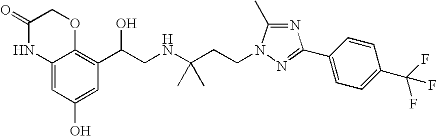

Prepared from 6-benzyloxy-8-(2-ethoxy-2-hydroxy-acetyl)-4H-benzo[1,4]oxazin-3-one and 1,1-dimethyl-3-[5-methyl-3-(4-trifluoromethyl-phenyl)-[1,2,4]triazol-1-yl]-propylamine analogously to General Method 1.

Yield: 234 mg (37%, trifluoroacetate); mass spectroscopy [M+H]⁺=520.

### Salt precursor 4: 8-(2-{3-[3-(3,5-difluoro-phenyl)-5-methyl-[1,2,4]triazol-1-yl]-1,1-dimethyl-propylamino}-1-hydroxy-ethyl)-6-hydroxy-4H-benzo[1,4]oxazin-3-one

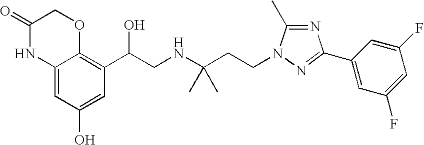

Prepared from 6-benzyloxy-8-(2-ethoxy-2-hydroxy-acetyl)-4H-benzo[1,4]oxazin-3-one and 3-[3-(3,5-difluoro-phenyl)-5-methyl-[1,2,4]triazol-1-yl]-1,1-dimethyl-propylamine according to General Method 1.

Yield: 208 mg (35%, trifluoroacetate); mass spectroscopy [M+H]⁺=488.

### Salt precursor 5: 3-(1-{3-[2-hydroxy-2-(6-hydroxy-3-oxo-3,4-dihydro-2H-benzo[1,4]oxazin-8-yl)-ethylamino]-3-methyl-butyl}-5-methyl-1H-[1,2,4]triazol-3-yl-benzoic acid

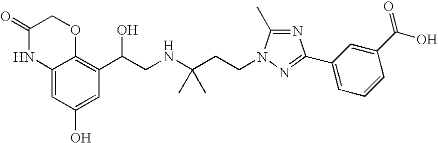

a) methyl 3-(1-{3-[2-(6-benzyloxy-3-oxo-3,4-dihydro-2H-benzo[1,4]oxazin-8-yl)-2-hydroxy-ethylamino]-3-methyl-butyl}-5-methyl-1H-[1,2,4]triazol-3-yl)-benzoate: prepared from 6-benzyloxy-8-(2-ethoxy-2-hydroxy-acetyl)-4H-benzo[1,4]oxazin-3-one and methyl 3-[1-(3-amino-3-methyl-butyl)-5-methyl-1H-[1,2,4]triazol-3-yl]-benzoate analogously to General Method 1. Final purification is carried out by chromatography (reverse phase, acetonitrile/water gradient with 0.1% trifluoroacetic acid).

Yield: 550 mg (77%, trifluoroacetate); mass spectroscopy [M+H]⁺=510.

b) 3(1-{3-[2-(6-benzyloxy-3-oxo-3,4-dihydro-2H-benzo[1,4]oxazin-8-yl)-2-hydroxy-ethylamino]-3-methyl-butyl}-5-methyl-1H-[1,2,4]triazol-3-yl)-benzoic acid: a solution of 550 mg (0.72 mmol) methyl 3-(1-{3-[2-(6-benzyloxy-3-oxo-3,4-dihydro-2H-benzo[1,4]oxazin-8-yl)-2-hydroxy-ethylamino]-3-methyl-butyl}-5-methyl-1H-[1,2,4]triazol-3-yl)-benzoate trifluoroacetate in 10 mL methanol is combined with 2 mL of a 2 molar sodium hydroxide solution and refluxed for 30 minutes. After the methanol has been distilled off 5 mL water, 10 mL n-butanol and 5 mL acetic acid are added. The precipitate formed is suction filtered and washed with diethyl ether.

Yield: 300 mg (56%, trifluoroacetate); mass spectroscopy [M+H]⁺=586.

c) 3-(1-{3-[2-hydroxy-2-(6-hydroxy-3-oxo-3,4-dihydro-2H-benzo[1,4]oxazin-8-yl)-ethylamino]-3-methyl-butyl}-5-methyl-1H-[1,2,4]triazol-3-yl-benzoic acid: 250 mg (0.36 mmol) 3-(1-{3-[2-(6-benzyloxy-3-oxo-3,4-dihydro-2H-benzo[1,4]oxazin-8-yl)-2-hydroxy-ethylamino]-3-methyl-butyl}-5-methyl-1H-[1,2,4]triazol-3-yl)-benzoic acid trifluoroacetate are dissolved in 5 mL methanol and hydrogenated in the presence of palladium on charcoal (10%) at ambient temperature and 2.5 bar hydrogen pressure. The catalyst is suction filtered, the filtrate is evaporated down and the residue is purified by chromatography (reverse phase, acetonitrile/water gradient with 0.1% trifluoroacetic acid).

Yield: 62 mg (28%, trifluoroacetate); mass spectroscopy [M+H]⁺=496.

### Salt precursor 6: 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

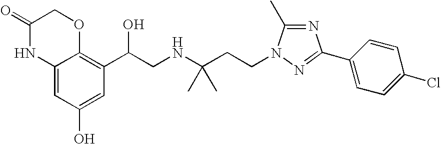

a) 6-benzyloxy-8-(2-{3-[3-(4-chloro-phenyl)-5-methyl-[1,2,4]triazol-1-yl]-1,1-dimethyl-propylamino}-1-hydroxy-ethyl)-4H-benzo[1,4]oxazin-3-one: prepared from 6-benzyloxy-8-(2-ethoxy-2-hydroxy-acetyl)-4H-benzo[1,4]oxazin-3-one and 3-[3-(4-chloro-phenyl)-5-methyl-[1,2,4]triazol-1-yl]-1,1-dimethyl-propyl amine analogously to General Method 1. The crude product is purified by chromatography (reverse phase, acetonitrile/water gradient with 0.1% trifluoroacetic acid).

Yield: 550 mg (80%, trifluoroacetate); mass spectroscopy [M+H]⁺=576.

8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one: 550 mg (0.80 mmol) 6-benzyloxy-8-(2-{3-[3-(4-chloro-phenyl)-5-methyl-[1,2,4]triazol-1-yl]-1,1-dimethyl-propylamino}-1-hydroxy-ethyl)-4H-benzo[1,4]oxazin-3-one are dissolved in 3 mL dichloromethane and cooled to 78° C. 2 mL of a 1 molar solution of boron tribromide in dichloromethane are added dropwise and the mixture is heated to ambient temperature. It is stirred for 10 minutes at this temperature and then 10 mL dichloromethane and 3 mL water are added and the mixture is stirred for 30 minutes. It is filtered through kieselguhr, while eluting with dichloromethane and methanol. The eluate is evaporated down and the residue is purified by chromatography (reverse phase, acetonitrile/water gradient with 0.1% trifluoroacetic acid).

Yield: 29 mg (6%, trifluoroacetate); mass spectroscopy [M+H]⁺=486/8.

### Salt precursor 7: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one

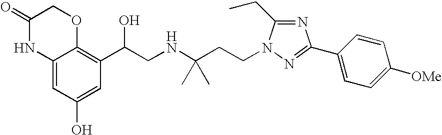

Prepared from 6-benzyloxy-8-(2-ethoxy-2-hydroxy-acetyl)-4H-benzo[1,4]oxazin-3-one and 3-[5-ethyl-3-(4-methoxy-phenyl)-[1,2,4]triazol-1-yl]-1,1-dimethyl-propylamine according to General Method 1.

Yield: 267 mg (44%, trifluoroacetate); mass spectroscopy [M+H]⁺=496.

### Salt precursor 8: 6-hydroxy-8-(1-hydroxy-2-{3-[3-(4-methoxy-phenyl)-5-methyl-[1,2,4]triazol-1-yl]-1,1-dimethyl-propylamino}-ethyl)-4H-benzo[1,4]oxazin-3-one

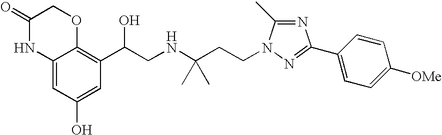

Prepared from 6-benzyloxy-8-(2-ethoxy-2-hydroxy-acetyl)-4H-benzo[1,4]oxazin-3-one and 3-[3-(4-methoxy-phenyl)-5-methyl-[1,2,4]triazol-1-yl]-1,1-dimethyl-propylamine according to General Method 2. Yield: 217 mg (45%); mass spectroscopy [M+H]⁺=482.

### Salt precursor 9: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one

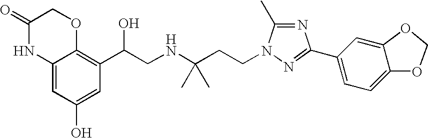

Prepared from 6-benzyloxy-8-(2-ethoxy-2-hydroxy-acetyl)-4H-benzo[1,4]oxazin-3-one and 3-(3-benzo[1,3]dioxol-5-yl-5-methyl-[1,2,4]triazol-1-yl)-1,1-dimethyl-propylamine according to General Method 2. Yield: 236 mg (48%); mass spectroscopy [M+H]⁺=496.

### Salt precursor 10: 7-{2-[3-(3-benzo[1,3]dioxol-5-yl-5-methyl-[1,2,4]triazol-1-yl)-1,1-dimethyl-propylamino]-1-hydroxy-ethyl}-5-hydroxy-3H-benzoxazol-2-one

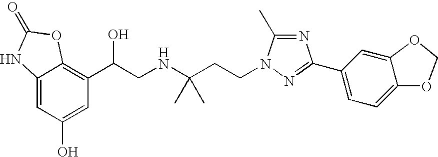

a) 7-acetyl-5-benzyloxy-3H-benzoxazol-2-one: 52 g (0.53 mol) phosgene are piped into a solution of 121 g (0.47 mol) 1-(3-amino-5-benzyloxy-2-hydroxy-phenyl)-ethanone in 800 mL pyridine at 20 to 40° C. The reaction mixture is heated to 50° C. for 2 hours, then poured onto ice and acidified with conc. hydrochloric acid. The precipitated solid is repeatedly recrystallised from ethanol with the addition of activated charcoal.

Yield: 67.5 g (51%); melting range: 163-166° C.

b) 5-benzyloxy-7-(2-ethoxy-2-hydroxy-acetyl)-3H-benzoxazol-2-one: 20 g (71 mmol) 7-acetyl-5-benzyloxy-3H-benzoxazol-2-one and 8 g (72 mmol) selenium dioxide are refluxed with stirring in the presence of activated charcoal in 100 mL dioxane and 3.1 mL water for 8 hours. The solid is filtered off, the solvent is distilled off and the residue is combined with 50 mL ethanol. The mixture is refluxed for 15 minutes and then filtered through activated charcoal. The solid that precipitates during cooling is suction filtered after 3 hours and washed with ethanol and diethyl ether.

Yield: 7 g (29%); melting range: 140-143° C.

c) 17-{2-[3-(3-benzo[1,3]dioxol-5-yl-5-methyl-[1,2,4]triazol-1-yl)-1,1-dimethyl-propylamino]-1-hydroxy-ethyl}-5-hydroxy-3H-benzoxazol-2-one: 72 mg (0 5 mmol) 5-benzyloxy-7-(2-ethoxy-2-hydroxy-acetyl)-3H-benzoxazol-2-one and 144 mg (0.5 mmol) 3-(3-benzo[1,3]dioxol-5-yl-5-methyl-[1,2,4]triazol-1-yl)-1,1-dimethyl-propylamine are stirred in 8 mL ethanol for 90 minutes at 80° C. After cooling to ambient temperature 19 mg (0.5 mmol) sodium borohydride are added and the mixture is stirred for 2 hours at ambient temperature. It is acidified with 1 N hydrochloric acid, stirred for 10 minutes and then made alkaline with potassium carbonate solution. It is diluted with ethyl acetate and filtered through kieselguhr. The organic phase remaining is evaporated down and the residue is purified by chromatography. The benzylether thus obtained is dissolved in ethanol and hydrogenated with palladium on charcoal (10%) as catalyst at 2.5 bar and ambient temperature. Then the catalyst is separated off and the crude product is purified by chromatography (reverse phase, acetonitrile/water gradient with 0.1% trifluoroacetic acid). Yield: 8 mg (3%, trifluoroacetate); mass spectroscopy [M+H]⁺=482.

### Salt precursor 11: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-2,2-dimethyl-4H-benzo[1,4]oxazin-3-one

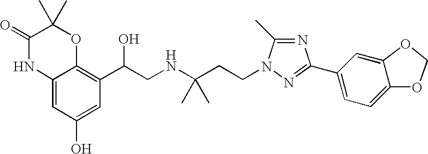

a) N-(3-acetyl-5-benzyloxy-2-hydroxy-phenyl)-2-bromo-2-methyl-propionamide: 4.64 g (25 mmol) 2-bromo-2-methyl-propionyl chloride are added dropwise at 5-20° C. to a solution of 5.15 g (20 mmol) 1-(3-amino-5-benzyloxy-2-hydroxy-phenyl)-ethanone in 20 mL pyridine. After the addition has ended the mixture is stirred for 15 minutes, combined with ice water and 100 mL ethyl acetate and acidified with conc. hydrochloric acid. The organic phase is separated off, washed with water and dried on sodium sulphate. After the solvent has been distilled off the residue is crystallised from a diethyl ether/petroleum ether mixture. Yield: 6.8 g (84%); melting range: 88-90° C.

b) 8-acetyl-6-benzyloxy-2,2-dimethyl-4H-benzo[1,4]oxazin-3-one: 6.60 g (16.2 mmol) N-(3-acetyl-5-benzyloxy-2-hydroxy-phenyl)-2-bromo-2-methyl-propionamide and 2.76 g (20 mmol) potassium carbonate are stirred for 1 hour in 70 mL acetonitrile at reflux temperature. The solid is suction filtered, the filtrate is evaporated down and the residue is combined with 30 mL ethyl acetate. After further filtration and distillation of the solvent the crude product is crystallised from a little methanol.

Yield: 1.00 g (19%); mass spectroscopy [M+H]⁺=326; melting range=148-150° C.

c) 6-benzyloxy-8-(2-ethoxy-2-hydroxy-acetyl)-2,2-dimethyl-4H-benzo[1,4]oxazin-3-one: This is prepared analogously to the method described for salt precursor 10b) from 8-acetyl-6-benzyloxy-2,2-dimethyl-4H-benzo[1,4]oxazin-3-one.

d) 8-{2-[3(3-benzo[1,3]dioxol-5-yl-5-methyl-[1,2,4]triazol-1-yl)-1,1-dimethyl-propylamino]-1-hydroxy-ethyl}-6-hydroxy-2,2-dimethyl-4H-benzo[1,4]oxazin-3-one: prepared from 385 mg (1 mmol) 6-benzyloxy-8-(2-ethoxy-2-hydroxy-acetyl)-2,2-dimethyl-4H-benzo[1,4]oxazin-3-one and 402 mg (1 mmol) 3-(3-benzo[1,3]dioxol-5-yl-5-methyl-[1,2,4]triazol-1-yl)-1,1-dimethyl-propylamine according to General Method 1.

Yield: 37 mg (6%, trifluoroacetate); mass spectroscopy [M+H]⁺=524.

The racemates described above may be resolved into the individual enantiomers in known manner.

### Examples of Salts

### Parameters of the X-Ray Powder Diffractometer used for the Measurement

STOE Stadi P X-ray powder diffractometer with location-sensitive detector in transmission mode with curved germanium (111) primary monochromator; wavelength used: CuK_{\acute{α}1} with λ=1.540598 Å; power absorption of the X-ray tube: 40 kV, 40 mA; absorption range: 3-40 °2Θ~.

The following Tables show the characteristic X-ray reflections with intensities (standardised, up to 30° 2Θ) for the Examples specified. The corresponding diagrams are also shown. As the skilled man knows, the intensities of the reflections may vary depending on the preparation of the samples. The intensities specified below were found on measuring the above-mentioned Example and cannot be transferred to any other measurement.

Technical data relating to the thermoanalytical DSC device used: DSC 822 made by Mettler Toledo; heating rate: 10 K/min; type of crucible: perforated aluminium crucible; atmosphere: N₂, 80 ml/min flux; typical weight: 3-10 mg.

Technical data relating to the thermoanalytical TG device used: TGA/SDTA 851 made by Mettler Toledo with IR coupling (Nicolet FT-IR 4700) for analysing the volatile fractions driven off; heating rate: 10 K/min; type of crucible: open aluminium oxide crucible; atmosphere: N₂, 20 ml/min flux; typical weight: 15-25 mg.

The melting points measured by DSC are stated in the Examples. The corresponding diagrams can be found in the Figures.

### Example 1

### 8-OR)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one benzoate

A solution of 125 mg (1.03 mmol) benzoic acid in 3 mL acetonitrile is added to a refluxing solution of 500 mg (1.03 mmol) 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one in 3 mL acetonitrile. Then the mixture is cooled to ambient temperature and then to 5° C. and stirred for 30 minutes at this temperature. The precipitate is filtered off, washed with acetonitrile and dried.

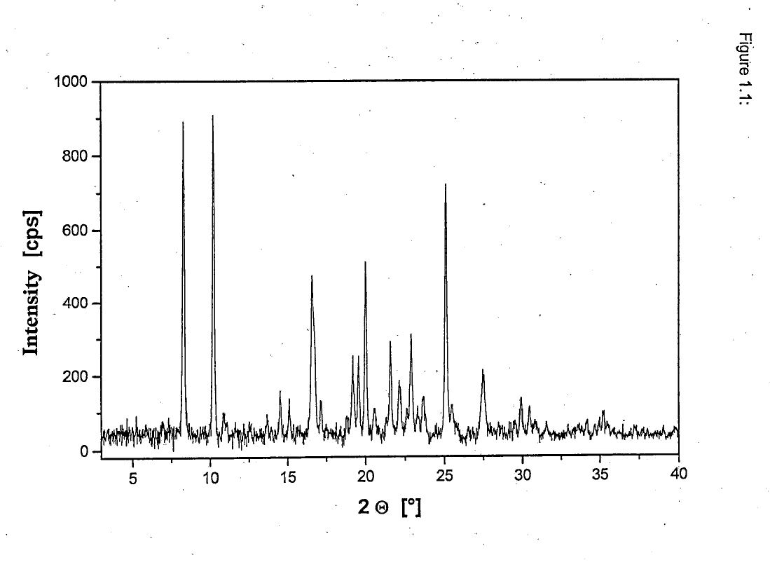

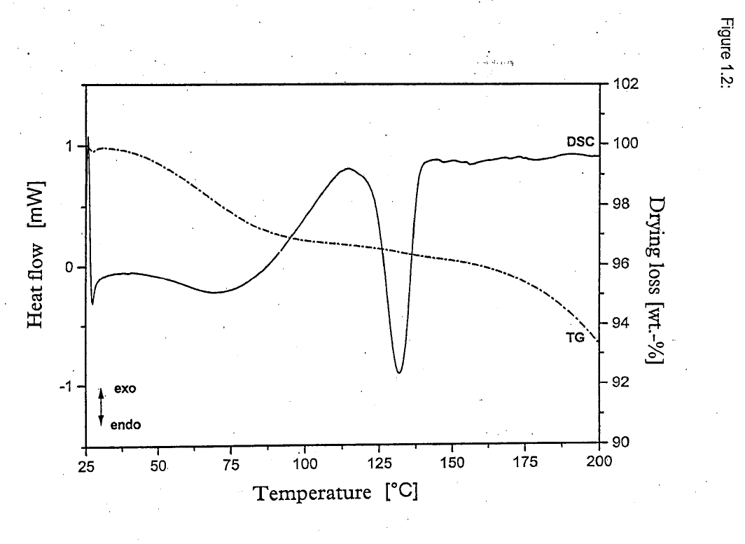

Yield: 490 mg (78%). Melting point according to DSC: 125±5° C. (see FIG. 1.2)

FIG. 1.1: X-ray powder diagram of the benzoate salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

FIG. 1.2: DSC/TG diagram of the benzoate salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

TABLE 1
X-ray reflections (up to 30° 2Θ) with intensities (standardised) of the
benzoate salt of 8-((R)-2-{3-[3-(4-chloro-phenyl)-5-methyl-
[1,2,4]triazol-1-yl]-1,1-dimethyl-propylamino}-1-hydroxy-ethyl)-
6-hydroxy-4H-benzo[1,4]oxazin-3-one

| 2Θ | dₕₖₗ | intensity |
|---|---|---|
| [°] | [Å] | I/Iₒ [%] |
| 8.32 | 10.62 | 98 |
| 10.25 | 8.62 | 100 |
| 10.88 | 8.12 | 8 |
| 12.55 | 7.05 | 6 |
| 13.70 | 6.46 | 8 |
| 14.52 | 6.10 | 14 |
| 15.12 | 5.86 | 10 |
| 16.59 | 5.34 | 52 |
| 16.76 | 5.28 | 30 |
| 17.14 | 5.17 | 12 |
| 19.20 | 4.62 | 25 |
| 19.57 | 4.53 | 24 |
| 20.04 | 4.43 | 53 |
| 20.57 | 4.32 | 9 |
| 21.61 | 4.11 | 31 |
| 22.17 | 4.01 | 18 |
| 22.62 | 3.93 | 8 |
| 22.91 | 3.88 | 31 |
| 23.31 | 3.81 | 10 |
| 23.68 | 3.75 | 13 |
| 25.11 | 3.54 | 79 |
| 25.50 | 3.49 | 11 |
| 27.47 | 3.24 | 20 |
| 29.92 | 2.98 | 12 |
| 30.46 | 2.93 | 10 |

### Example 2

### 8-OR)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one acetate

300 mg (0.62 mmol) 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one are suspended in 2 mL acetonitrile and heated to 70° C. 35 μl (0.62 mmol) acetic acid and a few drops of ethanol are added and the mixture is slowly cooled to ambient temperature. Then it is combined with more acetonitrile and a few drops of ethanol and heated to 50° C. The precipitate obtained after further cooling is filtered off and washed successively with acetonitrile and diethyl ether.

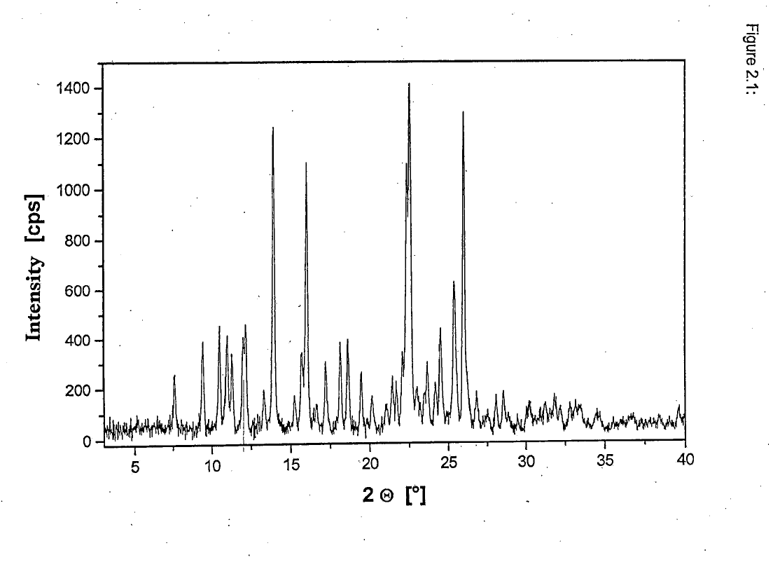

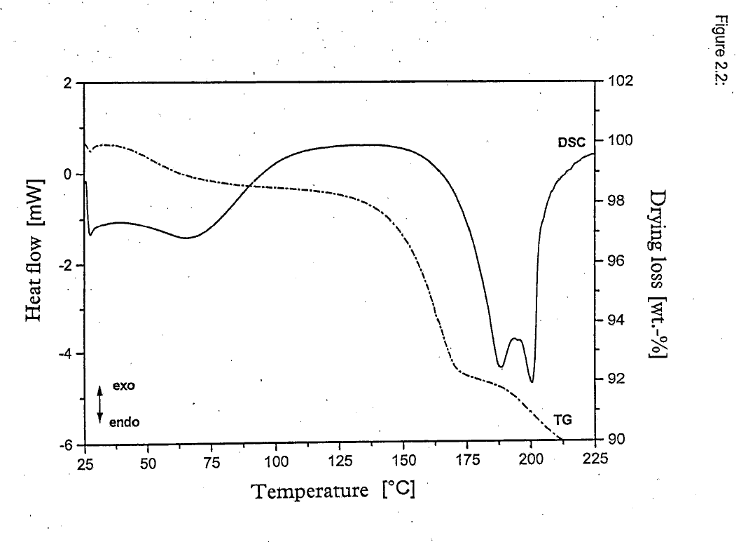

Yield: 291 mg (86%). Melting point according to DSC: approx. 160° C. with cleaving of acetic acid (see FIG. 2.2)

FIG. 2.1: X-ray powder diagram of the acetate salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

FIG. 2.2: DSC/TG diagram of the acetate salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

TABLE 2
X-ray reflections (up to 30° 2Θ) with intensities (standardised) of the
acetate salt of 8-((R)-2-{3-[3-(4-chloro-phenyl)-5-methyl-[1,2,4]triazol-
1-yl]-1,1-dimethyl-propylamino}-1-hydroxy-ethyl)-6-hydroxy-
4H-benzo[1,4]oxazin-3-one

| 2Θ | dₕₖₗ | intensity |
|---|---|---|
| [°] | [Å] | I/Iₒ [%] |
| 7.61 | 11.60 | 16 |
| 9.44 | 9.36 | 26 |
| 10.53 | 8.40 | 30 |
| 11.00 | 8.04 | 27 |
| 11.30 | 7.82 | 23 |
| 12.03 | 7.35 | 28 |
| 12.19 | 7.26 | 32 |
| 13.32 | 6.64 | 12 |
| 13.96 | 6.34 | 86 |
| 15.25 | 5.81 | 10 |
| 15.71 | 5.64 | 24 |
| 16.05 | 5.52 | 74 |
| 16.66 | 5.32 | 8 |
| 17.23 | 5.14 | 20 |
| 18.17 | 4.88 | 26 |
| 18.64 | 4.76 | 26 |
| 19.47 | 4.55 | 16 |
| 20.16 | 4.40 | 10 |
| 21.07 | 4.21 | 8 |
| 21.45 | 4.14 | 15 |
| 21.69 | 4.09 | 15 |
| 22.12 | 4.01 | 23 |
| 22.40 | 3.97 | 77 |
| 22.58 | 3.94 | 100 |
| 23.00 | 3.86 | 13 |
| 23.49 | 3.78 | 11 |
| 23.68 | 3.75 | 19 |
| 24.22 | 3.67 | 14 |
| 24.53 | 3.63 | 29 |
| 25.41 | 3.50 | 43 |
| 26.04 | 3.42 | 89 |
| 26.82 | 3.32 | 11 |
| 27.54 | 3.24 | 6 |
| 28.10 | 3.17 | 11 |
| 28.55 | 3.12 | 11 |
| 30.05 | 2.97 | 6 |

### Example 3

### 8-OR)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one L-lactate

56 mg (0.62 mmol) L(+)-lactic acid are added at ambient temperature to 300 mg (0.62 mmol) 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one in 2 mL of 2-propanol and the mixture is stirred for 2 hours. The resulting mixture is heated to 50° C., combined with a few drops of ethanol and slowly cooled. The precipitate is filtered off and washed successively with 2-propanol and diethyl ether.

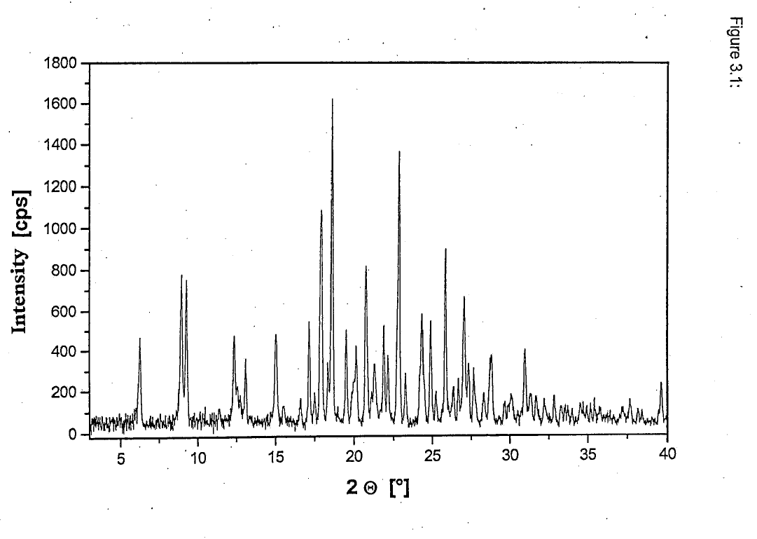

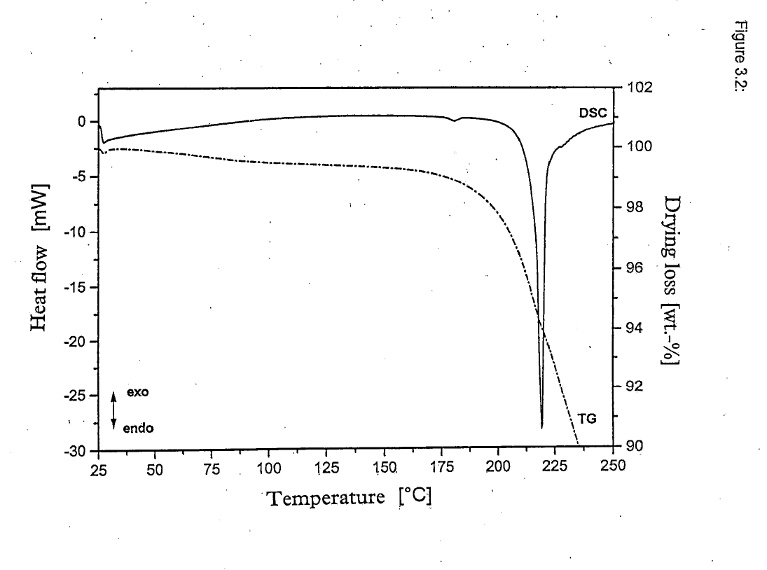

Yield: 287 mg (81%). Melting point according to DSC: 215±5° C. with decomposition (see FIG. 3.2)

FIG. 3.1: X-ray powder diagram of the L-lactate salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

FIG. 3.2: DSC/TG diagram of the L-lactate salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

TABLE 3
X-ray reflections (up to 30° 2Θ) with intensities (standardised) of the
L-lactate salt of 8-((R)-2-{3-[3-(4-chloro-phenyl)-5-methyl-
[1,2,4]triazol-1-yl]-1,1-dimethyl-propylamino}-1-hydroxy-ethyl)-
6-hydroxy-4H-benzo[1,4]oxazin-3-one

| 2Θ | dₕₖₗ | intensity |
|---|---|---|
| [°] | [Å] | I/Iₒ [%] |
| 7.44 | 11.88 | 4 |
| 9.46 | 9.34 | 13 |
| 10.33 | 8.56 | 8 |
| 10.90 | 8.11 | 24 |
| 11.38 | 7.77 | 27 |
| 11.66 | 7.59 | 25 |
| 13.02 | 6.80 | 7 |
| 13.92 | 6.36 | 4 |
| 14.76 | 6.00 | 67 |
| 15.62 | 5.67 | 8 |
| 15.98 | 5.54 | 71 |
| 16.82 | 5.27 | 13 |
| 18.30 | 4.84 | 9 |
| 18.84 | 4.71 | 18 |
| 19.61 | 4.52 | 36 |
| 20.37 | 4.36 | 9 |
| 20.65 | 4.30 | 10 |
| 21.31 | 4.17 | 100 |
| 21.91 | 4.05 | 21 |
| 22.50 | 3.95 | 12 |
| 22.86 | 3.89 | 27 |
| 23.10 | 3.85 | 12 |
| 23.85 | 3.73 | 20 |
| 23.99 | 3.71 | 20 |
| 24.25 | 3.67 | 23 |
| 24.63 | 3.61 | 58 |
| 24.88 | 3.58 | 34 |
| 26.05 | 3.42 | 18 |
| 26.42 | 3.37 | 9 |
| 26.84 | 3.32 | 6 |
| 27.15 | 3.28 | 9 |
| 29.71 | 3.01 | 6 |

### Example 4

### 8-OR)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one maleate

300 mg (0.62 mmol) 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one in 2 mL of 2-propanol are combined at 65° C. with 72 mg (0.62 mmol) maleic acid. The mixture is stirred for 1 hour and slowly cooled. The resulting precipitate is filtered off and washed successively with 2-propanol and diethyl ether.

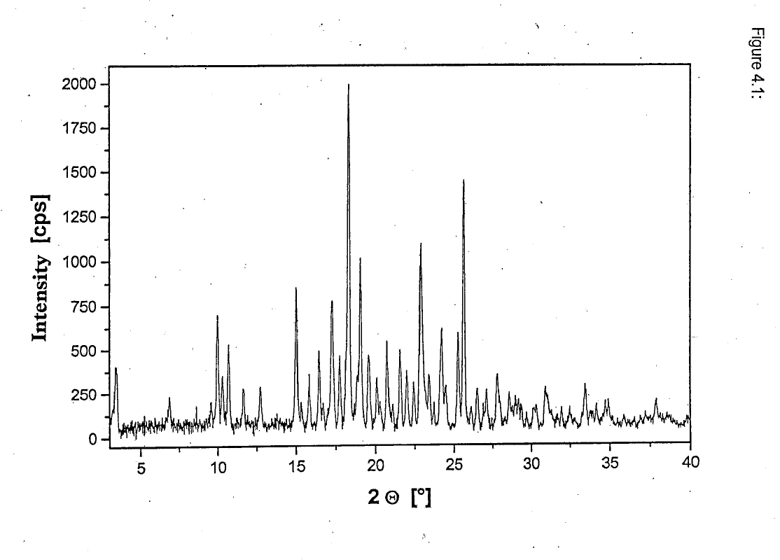

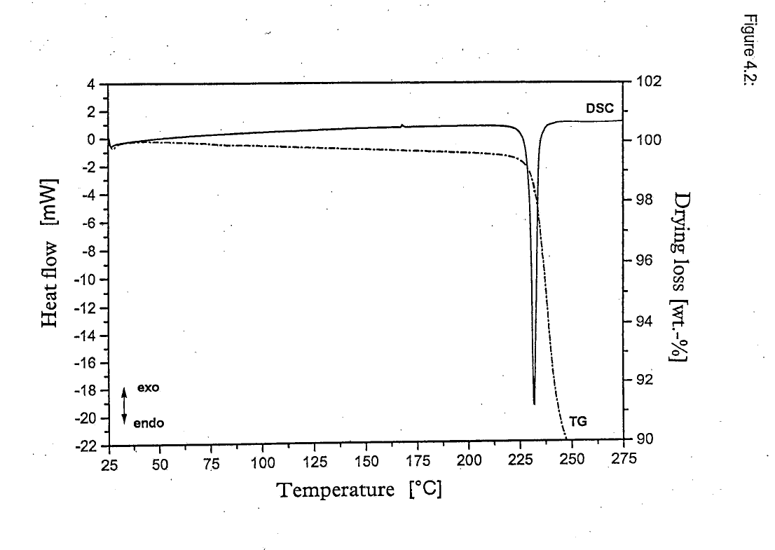

Yield: 275 mg (74%). Melting point according to DSC: 230±5° C. with decomposition (see FIG. 4.2)

FIG. 4.1: X-ray powder diagram of the maleate salt of 8-((R)-2-{3-[3-(4-chlorophenyl)-5-methyl-[1,2,4]triazol-1-yl]-1,1-dimethyl-propylamino}-1-hydroxy-ethyl)-6-hydroxy-4H-benzo[1,4]oxazin-3-one

FIG. 4.2: DSC/TG diagram of the maleate salt of 8-((R)-2-{3-[3-(4-chlorophenyl)-5-methyl-[1,2,4]triazol-1-yl]-1,1-dimethyl-propylamino}-1-hydroxy-ethyl)-6-hydroxy-4H-benzo[1,4]oxazin-3-one

TABLE 4
X-ray reflections (up to 30° 2Θ) with intensities (standardised) of the
maleate salt of 8-((R)-2-{3-[3-(4-chloro-phenyl)-5-methyl-
[1,2,4]triazol-1-yl]-1,1-dimethyl-propylamino}-1-hydroxy-
ethyl)-6-hydroxy-4H-benzo[1,4]oxazin-3-one

| 2Θ | dₕₖₗ | intensity |
|---|---|---|
| [°] | [Å] | I/Iₒ [%] |
| 11.66 | 7.59 | 13 |
| 12.76 | 6.93 | 13 |
| 13.79 | 6.42 | 4 |
| 15.05 | 5.88 | 42 |
| 15.32 | 5.78 | 8 |
| 15.83 | 5.59 | 14 |
| 16.47 | 5.38 | 24 |
| 16.72 | 5.30 | 8 |
| 17.29 | 5.13 | 40 |
| 17.78 | 4.99 | 21 |
| 18.36 | 4.83 | 100 |
| 18.89 | 4.70 | 16 |
| 19.08 | 4.65 | 50 |
| 19.60 | 4.53 | 22 |
| 20.11 | 4.41 | 16 |
| 20.31 | 4.37 | 8 |
| 20.77 | 4.27 | 26 |
| 21.11 | 4.21 | 7 |
| 21.60 | 4.11 | 24 |
| 22.01 | 4.04 | 17 |
| 22.45 | 3.96 | 15 |
| 22.91 | 3.88 | 54 |
| 23.42 | 3.80 | 17 |
| 24.24 | 3.67 | 29 |
| 24.50 | 3.63 | 13 |
| 25.29 | 3.52 | 29 |
| 25.67 | 3.47 | 74 |
| 26.13 | 3.41 | 6 |
| 26.55 | 3.35 | 12 |
| 26.90 | 3.31 | 6 |
| 27.12 | 3.29 | 12 |
| 27.83 | 3.20 | 16 |
| 28.60 | 3.12 | 11 |
| 28.98 | 3.08 | 8 |
| 29.18 | 3.06 | 8 |
| 29.37 | 3.04 | 7 |
| 29.71 | 3.00 | 4 |
| 11.66 | 7.59 | 13 |
| 12.76 | 6.93 | 13 |
| 13.79 | 6.42 | 4 |
| 15.05 | 5.88 | 42 |
| 15.32 | 5.78 | 8 |
| 15.83 | 5.59 | 14 |

### Example 5

### 8-OR)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one malate

83 mg (0.62 mmol) L(−)-malic acid are added to 300 mg (0.62 mmol) 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one in 2 mL of 2-propanol, whereupon a precipitate is formed. The mixture is heated to 50° C. and 2-propanol and a few drops of ethanol are added until a clear solution is obtained. Then it is left to cool slowly. The precipitate is filtered off, washed successively with 2-propanol and diethyl ether and dried.

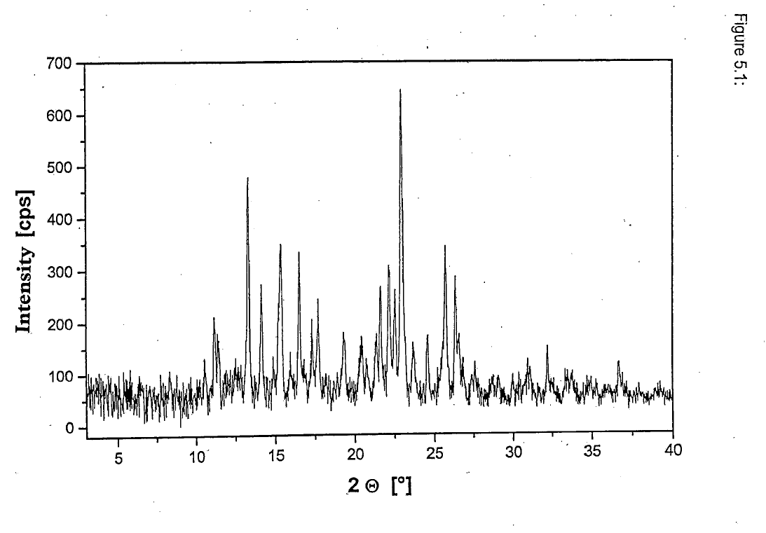

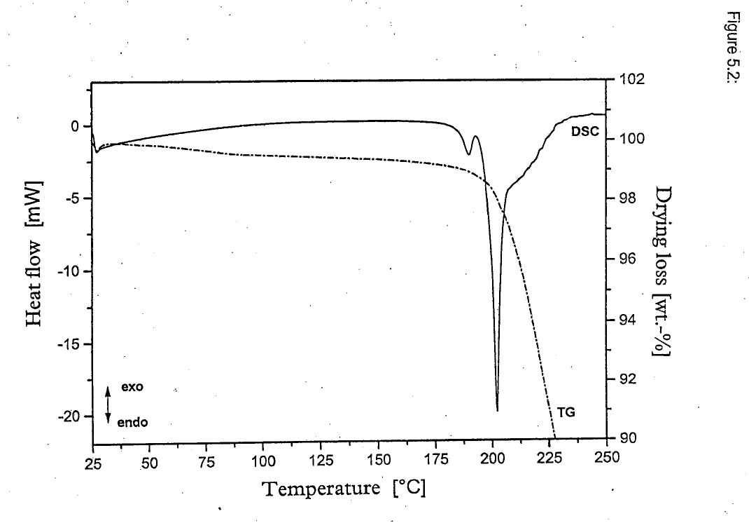

Yield: 277 mg (72%). Melting point according to DSC: 200±5° C. with decomposition (see FIG. 5.2)

FIG. 5.1: X-ray powder diagram of the malate salt of 8-((R)-2-{3-[3-(4-chlorophenyl)-5-methyl-[1,2,4]triazol-1-yl]-1,1-dimethyl-propylamino}-1-hydroxy-ethyl)-6-hydroxy-4H-benzo[1,4]oxazin-3-one

FIG. 5.2: DSC/TG diagram of the malate salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

TABLE 5
X-ray reflections (up to 30° 2Θ) with intensities (standardised) of the
malate salt of 8-((R)-2-{3-[3-(4-chloro-phenyl)-5-methyl-
[1,2,4]triazol-1-yl]-1,1-dimethylpropylamino}-1-hydroxy-ethyl)-
6-hydroxy-4H-benzo[1,4]oxazin-3-one

| 2Θ | dₕₖₗ | intensity |
|---|---|---|
| [°] | [Å] | I/Iₒ [%] |
| 8.34 | 10.59 | 12 |
| 10.57 | 8.36 | 16 |
| 11.19 | 7.90 | 28 |
| 11.44 | 7.73 | 18 |
| 12.54 | 7.05 | 15 |
| 13.35 | 6.63 | 75 |
| 14.16 | 6.25 | 38 |
| 14.89 | 5.94 | 13 |
| 15.37 | 5.76 | 50 |
| 15.98 | 5.54 | 16 |
| 16.54 | 5.35 | 48 |
| 17.32 | 5.11 | 25 |
| 17.72 | 5.00 | 32 |
| 19.31 | 4.59 | 23 |
| 20.48 | 4.33 | 21 |
| 20.76 | 4.28 | 15 |
| 21.37 | 4.15 | 23 |
| 21.64 | 4.10 | 39 |
| 22.17 | 4.01 | 44 |
| 22.55 | 3.94 | 37 |
| 22.97 | 3.87 | 100 |
| 23.69 | 3.75 | 19 |
| 24.61 | 3.61 | 21 |
| 25.74 | 3.46 | 49 |
| 26.37 | 3.38 | 41 |
| 26.59 | 3.35 | 21 |
| 26.85 | 3.32 | 9 |
| 27.35 | 3.26 | 8 |
| 27.60 | 3.23 | 11 |
| 28.67 | 3.11 | 9 |
| 29.10 | 3.07 | 9 |

### Example 6

### 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one hydrobromide dihydrate

166 μL 30% hydrobromide in acetic acid are added at 65° C. to a solution of 300 mg (0.62 mmol) 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one in 2 mL ethanol. The mixture is stirred for one hour at this temperature and then slowly cooled. The precipitate formed is separated off and washed successively with ethanol and diethyl ether.

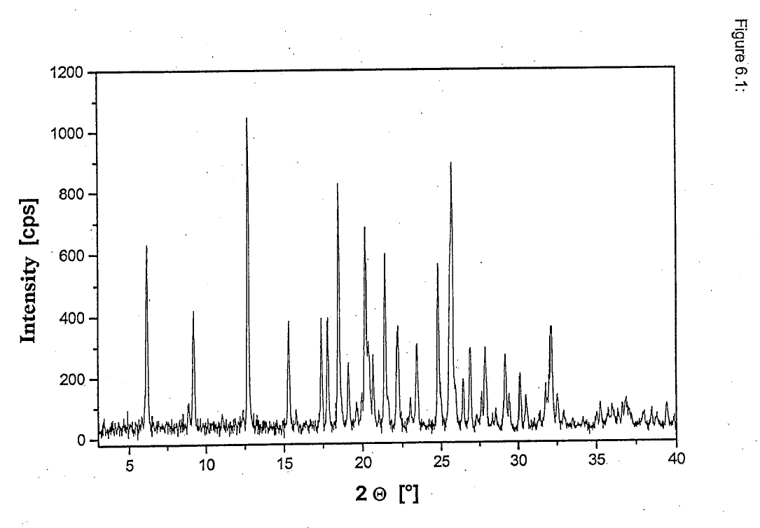

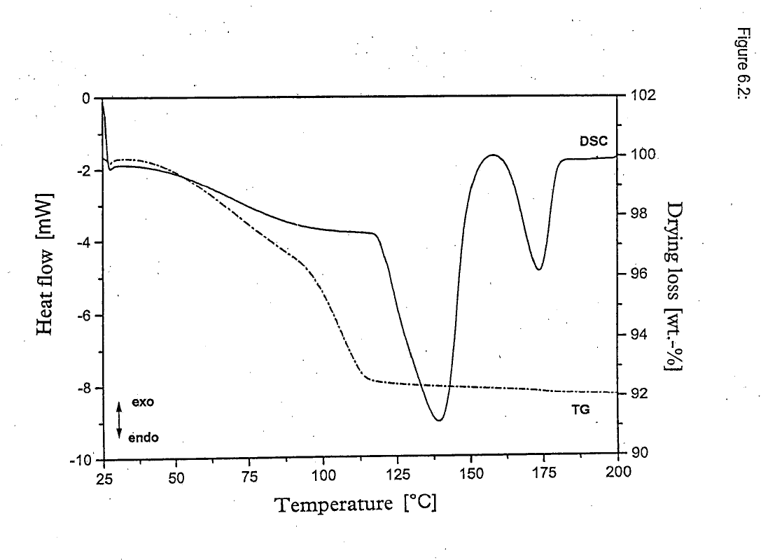

Yield: 226 mg. Melting point according to DSC: 165±5° C. (see FIG. 6.2)

FIG. 6.1: X-ray powder diagram of the bromide salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

FIG. 6.2: DSC/TG diagram of the bromide salt of 8-((R)-2-{3-[3-(4-chlorophenyl)-5-methyl-[1,2,4]triazol-1-yl]-1,1-dimethyl-propylamino}-1-hydroxy-ethyl)-6-hydroxy-4H-benzo[1,4]oxazin-3-one

TABLE 6
X-ray reflections (up to 30° 2Θ) with intensities (standardised) of the
bromide salt of 8-((R)-2-{3-[3-(4-chloro-phenyl)-5-methyl-
[1,2,4]triazol-1-yl]-1,1-dimethyl-propylamino}-1-hydroxy-ethyl)-
6-hydroxy-4H-benzo[1,4]oxazin-3-one

| 2Θ | dₕₖₗ | intensity |
|---|---|---|
| [°] | [Å] | I/Iₒ [%] |
| 6.20 | 14.25 | 57 |
| 8.88 | 9.96 | 9 |
| 9.21 | 9.59 | 36 |
| 11.06 | 8.00 | 6 |
| 12.40 | 7.13 | 6 |
| 12.73 | 6.95 | 100 |
| 15.31 | 5.78 | 34 |
| 15.77 | 5.61 | 6 |
| 17.40 | 5.09 | 35 |
| 17.82 | 4.97 | 35 |
| 18.48 | 4.80 | 76 |
| 19.10 | 4.64 | 22 |
| 19.60 | 4.52 | 8 |
| 19.95 | 4.45 | 10 |
| 20.19 | 4.39 | 64 |
| 20.36 | 4.36 | 26 |
| 20.65 | 4.30 | 23 |
| 21.43 | 4.14 | 53 |
| 21.61 | 4.11 | 11 |
| 22.22 | 4.00 | 31 |
| 23.02 | 3.86 | 9 |
| 23.45 | 3.79 | 27 |
| 24.81 | 3.59 | 51 |
| 24.96 | 3.56 | 15 |
| 25.61 | 3.48 | 54 |
| 25.72 | 3.46 | 79 |
| 25.94 | 3.43 | 14 |
| 26.43 | 3.37 | 16 |
| 26.89 | 3.31 | 27 |
| 27.64 | 3.22 | 12 |
| 27.89 | 3.20 | 26 |
| 28.32 | 3.15 | 3 |
| 28.54 | 3.12 | 7 |
| 29.18 | 3.06 | 23 |
| 29.42 | 3.03 | 11 |
| 30.14 | 2.96 | 18 |

### Example 7

### 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one hydrochloride dihydrate

411 μL hydrochloride in ethanol (1.25 M) are added at 65° C. to a solution of 250 mg (0.51 mmol) 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one in 2 mL 2-propanol. The mixture is stirred for one hour at this temperature and then slowly cooled to ambient temperature. The precipitate formed is filtered off and washed successively with 2-propanol and diethyl ether washed.

Yield: 259 mg.

200 mg of the solid from the above experiment are suspended in 1 mL acetonitrile and heated to 60° C. More acetonitrile is added and a few drops of water are added. The resulting clear solution is slowly cooled to ambient temperature and the precipitate formed is separated off and washed successively with acetonitrile and diethyl ether. Yield: 95 mg.

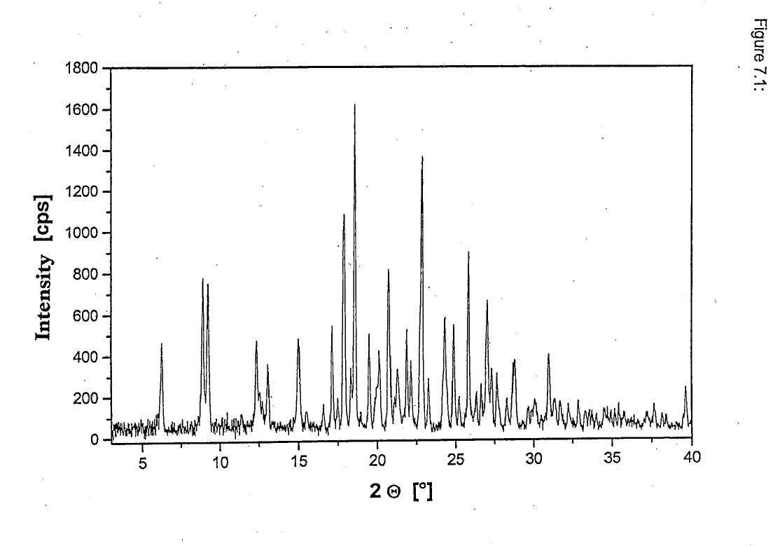

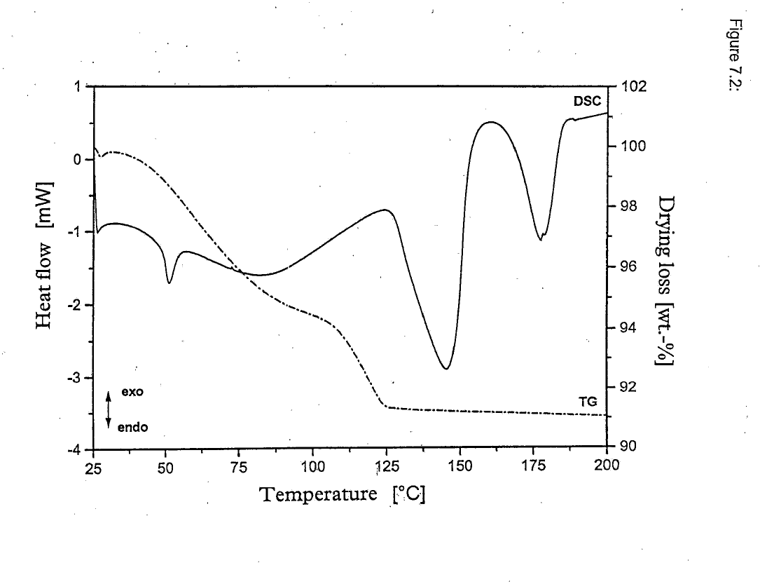

FIG. 7.1: X-ray powder diagram of the chloride salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

FIG. 7.2: DSC/TG diagram of the chloride salt of 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one

TABLE 7
X-ray reflections (up to 30° 2Θ) with intensities (standardised) of the
chloride salt of 8-((R)-2-{3-[3-(4-chloro-phenyl)-5-methyl-
[1,2,4]triazol-1-yl]-1,1-dimethyl-propylamino}-1-hydroxy-ethyl)-
6-hydroxy-4H-benzo[1,4]oxazin-3-one

| 2Θ | dₕₖₗ | intensity |
|---|---|---|
| [°] | [Å] | I/Iₒ [%] |
| 6.28 | 14.06 | 28 |
| 8.96 | 9.86 | 48 |
| 9.28 | 9.53 | 46 |
| 11.39 | 7.76 | 5 |
| 12.38 | 7.15 | 29 |
| 12.58 | 7.03 | 12 |
| 12.74 | 6.94 | 10 |
| 13.09 | 6.76 | 20 |
| 15.04 | 5.89 | 29 |
| 15.51 | 5.71 | 6 |
| 16.60 | 5.34 | 8 |
| 17.16 | 5.16 | 31 |
| 17.51 | 5.06 | 10 |
| 17.93 | 4.94 | 69 |
| 18.36 | 4.83 | 18 |
| 18.62 | 4.76 | 100 |
| 19.51 | 4.55 | 29 |
| 19.98 | 4.44 | 14 |
| 20.16 | 4.40 | 24 |
| 20.76 | 4.27 | 50 |
| 21.12 | 4.20 | 10 |
| 21.31 | 4.17 | 19 |
| 21.90 | 4.05 | 30 |
| 22.17 | 4.01 | 22 |
| 22.87 | 3.89 | 83 |
| 23.28 | 3.82 | 15 |
| 24.23 | 3.67 | 16 |
| 24.35 | 3.65 | 35 |
| 24.48 | 3.63 | 13 |
| 24.90 | 3.57 | 33 |
| 25.24 | 3.53 | 9 |
| 25.86 | 3.44 | 54 |
| 26.36 | 3.38 | 11 |
| 26.68 | 3.34 | 14 |
| 27.06 | 3.29 | 40 |
| 27.33 | 3.26 | 19 |
| 27.68 | 3.22 | 17 |
| 27.82 | 3.20 | 6 |
| 28.32 | 3.15 | 9 |
| 28.73 | 3.11 | 19 |
| 28.81 | 3.10 | 22 |
| 29.67 | 3.01 | 7 |

The compounds named below may be prepared analogously to the above mentioned methods, while in each case the enantiomerically pure compounds in the R-form are preferred:
1.2a: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one hydrochloride;
1.3a: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one hydrochloride;
1.7a: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one hydrochloride;
1.9a: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one hydrochloride;
1.2b: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one hydrobromide;
1.3b: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one hydrobromide;
1.7b: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one hydrobromide;
1.9b: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one hydrobromide;
1.2c: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one malate;
1.3c: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one malate;
1.7c: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one malate;
1.9c: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one malate;
1.2d: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one maleate;
1.3d: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one maleate;
1.7d: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one maleate;
1.9d: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one maleate;
1.2e: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one L-lactate;
1.3e: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one L-lactate;
1.7e: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one L-lactate;
1.9e: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one L-lactate.

### Combinations

The compounds of formula 1 may be used on their own or in combination with other active substances of formula 1. If desired the compounds of formula 1 may also be used in combination with W, where W denotes a pharmacologically active substance and (for example) is selected from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors. Moreover, double or triple combinations of W may be combined with the compounds of formula 1. Combinations of W might be, for example:
W denotes a betamimetic, combined with an anticholinergic, corticosteroid, PDE4-inhibitor, EGFR-inhibitor or LTD4-antagonist,
W denotes an anticholinergic, combined with a betamimetic, corticosteroid, PDE4-inhibitor, EGFR-inhibitor or LTD4-antagonist,
W denotes a corticosteroid, combined with a PDE4-inhibitor, EGFR-inhibitor or LTD4-antagonist
W denotes a PDE4-inhibitor, combined with an EGFR-inhibitor or LTD4-antagonist
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5.6-diethyl-indan-2-ylamino)-1-hydroxy-ethyl]-8-hydroxy-1H-quinoline-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl)-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazole-3-yl]-2-methyl-2-butylamino}ethanol
5-hydroxy-8-(1-hydroxy-2-isopropylaminobutyl)-2H-1,4-benzoxazin-3-(4H)-on
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2.4.6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl)-1,1-dimethyl-ethylamino]-ethyl}-4H-benzo[1,4]oxazin-3-one
6-hydroxy-8-{1-hydroxy-2-[2-(4-isopropyl-phenyl)-1.1dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3.4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
2-Hydroxy-5-(1-hydroxy-2-{2-[4-(2-hydroxy-2-phenyl-ethylamino)-phenyl]-ethylamino}-ethyl)-benzaldehyde
8-Hydroxy-5-(1-hydroxy-2-{2-[4-(6-methoxy-biphenyl-3-ylamino)-phenyl]-ethylamino}-ethyl)-1H-quinolin-2-one
8-Hydroxy-5-[1-hydroxy-2-(6-phenethylamino-hexylamino)-ethyl]-1H-quinolin-2-one
5-[2-(2-{-4-[4-(2-amino-2-methyl-propoxy)-phenylamino]-phenyl}-ethylamino)-1-hydroxy-ethyl]-8-hydroxy-1H-quinolin-2-one
[3-(4-{6-[2-hydroxy-2-(4-hydroxy-3-hydroxymethyl-phenyl)-ethylamino]-hexyloxy}-butyl)-5-methyl-phenyl]-urea
4-(2-{6-[2-(2,6-dichloro-benzyloxy)-ethoxy]-hexylamino}-1-hydroxy-ethyl)-2-hydroxymethyl-phenol
2-hydroxymethyl-4-{1-hydroxy-2-[6-(4-m-tolyl-butoxy)-hexylamino]-ethyl}-phenol
2-hydroxymethyl-4-{1-hydroxy-2-[7-(3-m-tolyl-propoxy)-heptylamino]-ethyl}-phenol
4-(2-{6-[4-(3-cyclopentanesulphonyl-phenyl)-butoxy]-hexylamino}-1-hydroxy-ethyl)-2-hydroxymethyl-phenol
N-adamantan-2-yl-2-(3-{2-[2-hydroxy-2-(4-hydroxy-3-hydroxymethyl-phenyl)-ethylamino]-propyl}-phenyl)-acetamide,
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other preferred anticholinergics are selected from among the salts of formula AC-1

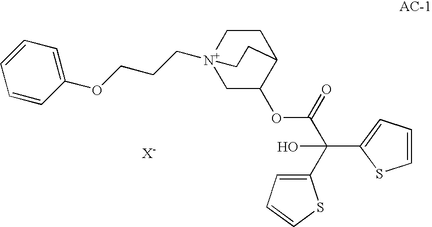

wherein X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-ene

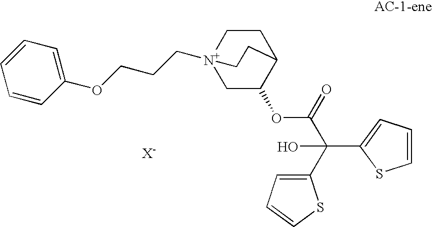

wherein X⁻ may have the above-mentioned meanings. Other preferred anticholinergics are selected from the salts of formula AC-2

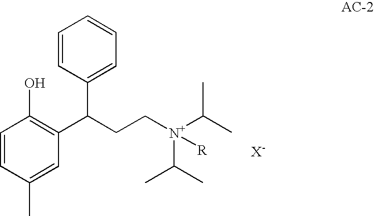

wherein R denotes either methyl or ethyl and wherein X⁻ may have the above-mentioned meanings. In an alternativen embodiment the compound of formula AC-2 may also be present in the form of the free base AC-2-base.

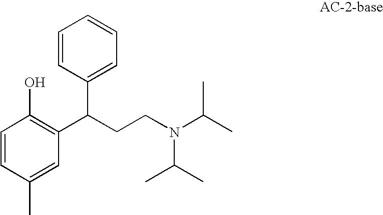

Other specified compounds are:
tropenol 2,2-diphenylpropionate methobromide,
scopine 2,2-diphenylpropionate methobromide,
scopine 2-fluoro-2,2-diphenylacetate methobromide,
tropenol 2-fluoro-2,2-diphenylacetate methobromide;
tropenol 3,3′,4,4′-tetrafluorobenzilate methobromide.
scopine 3,3′,4,4′-tetrafluorobenzilate methobromide,
tropenol 4,4′-difluorobenzilate methobromide,
scopine 4,4′-difluorobenzilate methobromide,
tropenol 3,3′-difluorobenzilate methobromide.
scopine 3,3′-difluorobenzilate methobromide;
tropenol 9-hydroxy-fluorene-9-carboxylate methobromide;
tropenol 9-fluoro-fluorene-9-carboxylate methobromide;
scopine 9-hydroxy-fluorene-9-carboxylate methobromide;
scopine 9-fluoro-fluorene-9-carboxylate methobromide;
tropenol 9-methyl-fluorene-9-carboxylate methobromide;
scopine 9-methyl-fluorene-9-carboxylate methobromide;
cyclopropyltropine benzilate methobromide;
cyclopropyltropine 2,2-diphenylpropionate methobromide;
cyclopropyltropine 9-hydroxy-xanthene-9-carboxylate methobromide;
cyclopropyltropine 9-methyl-fluorene-9-carboxylate methobromide;
cyclopropyltropine 9-methyl-xanthene-9-carboxylate methobromide;
cyclopropyltropine 9-hydroxy-fluorene-9-carboxylate methobromide;
cyclopropyltropine methyl 4,4′-difluorobenzilate methobromide.
tropenol 9-hydroxy-xanthene-9-carboxylate methobromide;
scopine 9-hydroxy-xanthene-9-carboxylate methobromide;
tropenol 9-methyl-xanthene-9-carboxylate-methobromide;
scopine 9-methyl-xanthene-9-carboxylate-methobromide;
tropenol 9-ethyl-xanthene-9-carboxylate methobromide;
tropenol 9-difluoromethyl-xanthene-9-carboxylate methobromide;
scopine 9-hydroxymethyl-xanthene-9-carboxylate methobromide,

The above-mentioned compounds may also be used as salts within the scope of the present invention, wherein instead of the methobromide the salts metho-X are used, wherein X may have the meanings given hereinbefore for X⁻.

As corticosteroids it is preferable to use compounds selected from among prednisolone, prednisone, butixocort propionate, flunisolide, beclomethasone, triamcinolone, budesonide, fluticasone, mometasone, ciclesonide, rofleponide, dexamethasone, betamethasone, deflazacort, RPR-106541, NS-126, ST-26 and
(S)-fluoromethyl 6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl)6,9-difluoro-11-hydroxy-16-methyl-3-oxo-17-propionyloxy-androsta-1,4-diene-17β-carbothionate,
cyanomethyl 6\acute{α},9\acute{α}-difluoro-11β-hydroxy-16\acute{α}-methyl-3-oxo-17\acute{α}-(2,2,3,3-tetramethylcyclopropylcarbonyl)oxy-androsta-1,4-diene-17β-carboxylate
etiprednol-dichloroacetate
   optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, CI-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)-p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N-2-cyano-5-methyl-isothioureido]benzyl)-2-pyrrolidone
cis[4-cyano-4-(3-cyclopentyloxy-4-methoxyphenyl)cyclohexane-1-carboxylic acid]
2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3.4-c]-1,2,4-triazolo[4.3-a]pyridine
9-cyclopentyl-5,6-dihydro-7-ethyl-3-(tert-butyl)-9H-pyrazolo[3.4-c]-1,2,4-triazolo[4.3-a]pyridine
   optionally in the form of the racemates, enantiomers or diastereomers thereof and optionally in the form of the pharmacologically acceptable acid addition salts thereof, the solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid
[2-[[2-(4-tert-butyl-2-thiazolyl)-5-benzofuranyl]oxymethyl]phenyl]acetic acid
   optionally in the form of the racemates, enantiomers or diastereomers thereof and optionally in the form of the pharmacologically acceptable acid addition salts, solvates and/or hydrates thereof. According to the invention the acid addition salts of the betamimetics are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

EGFR-inhibitors which may be used are preferably compounds selected from among cetuximab, trastuzumab, ABX-EGF, Mab ICR-62 and
4-[(3-chloro-4-fluorophenyl)amino]-6-{[4-(morpholin-4-yl)-1-oxo-2-buten-1-yl]-amino}-7-cyclopropylmethoxy-quinazoline
4-[(3-chloro-4-fluorophenyl)amino]-6-{[4-(N,N-diethylamino)-1-oxo-2-buten-1-yl]-amino}-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopropylmethoxy-quinazoline
4-[(R)-(1-phenyl-ethyl)amino]-6-{[4-(morpholin-4-yl)-1-oxo-2-buten-1-yl]amino}-7-cyclopentyloxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline
4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline
4-[(3-chloro-4-fluoro-phenyl)amino]-6-[2-((S)-6-methyl-2-oxo-morpholin-4-yl)-ethoxy]-7-methoxy-quinazoline
4-[(3-chloro-4-fluorophenyl)amino]-6-({-4-[N-(2-methoxy-ethyl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-cyclopentyloxy-quinazoline
4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline
4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline
4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline
4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline
4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline
4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline
4-[(3-chloro-4-fluorophenyl)amino]-6-({-44N-(2-methoxy-ethyl)-N-methyl-amino)-1-oxo-2-buten-1-yl}amino)-7-cyclopentyloxy-quinazoline
4-[(3-chloro-4-fluorophenyl)amino]-6-{[4-(N-cyclopropyl-N-methyl-amino)-1-oxo-2-buten-1-yl]amino}-7-cyclopentyloxy-quinazoline
4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline
4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline
4-[(3-ethynyl-phenyl)amino]-6.7-bis-(2-methoxy-ethoxy)-quinazoline
4-[(3-chloro-4-fluorophenyl)amino]-7-[3-(morpholin-4-yl)-propyloxy]-6-[(vinyl-carbonyl)amino]-quinazoline
4-[(R)-(1-phenyl-ethyl)amino]-6-(4-hydroxy-phenyl)-7H-pyrrolo[2,3-d]pyrimidine
3-cyano-4-[(3-chloro-4-fluorophenyl)amino]-6-{[4-(N,N-dimethylamino)-1-oxo-2-buten-1-yl]amino}-7-ethoxy-quinoline
4-{[3-chloro-4-(3-fluoro-benzyloxy)-phenyl]amino}-6-(5-{[(2-methanesulphonyl-ethyl)amino]methyl}-furan-2-yl)quinazoline
4-[(R)-(1-phenyl-ethyl)amino]-6-{[4-((R)-6-methyl-2-oxo-morpholin-4-yl)-1-oxo-2-buten-1-yl]amino}-7-methoxy-quinazoline
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline
4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline
4-[(3-ethynyl-phenyl)amino]-6-{[4-(5 5-dimethyl-2-oxo-morpholin-4-yl)-1-oxo-2-buten-1-yl]amino}-quinazoline
4-[(3-chloro-4-fluoro-phenyl)amino]-6-[2-(2.2-dimethyl-6-oxo-morpholin-4-yl)-ethoxy]-7-methoxy-quinazoline
4-[(3-chloro-4-fluoro-phenyl)amino]-6-[2-(2.2-dimethyl-6-oxo-morpholin-4-yl)-ethoxy]-7-[(R)-(tetrahydrofuran-2-yl)methoxy]-quinazoline
4-[(3-chloro-4-fluoro-phenyl)amino]-7-[2-(2.2-dimethyl-6-oxo-morpholin-4-yl)-ethoxy]-6-[(S)-(tetrahydrofuran-2-yl)methoxy]-quinazoline
4-[(3-chloro-4-fluoro-phenyl)amino]-6-{2-[4-(2-oxo-morpholin-4-yl)-piperidin-1-yl]-ethoxy}-7-methoxy-quinazoline
4-[(3-chloro-4-fluoro-phenyl)amino]-6-[1-(tert.-butyloxycarbonyl)-piperidin-4-yloxy]-7-methoxy-quinazoline
4-[(3-chloro-4-fluoro-phenyl)amino]-6-(trans-4-amino-cyclohexan-1-yloxy)-7-methoxy-quinazoline
4-[(3-chloro-4-fluoro-phenyl)amino]-6-(trans-4-methanesulphonylamino-cyclohexan-1-yloxy)-7-methoxy-quinazoline
4-[(3-chloro-4-fluoro-phenyl)amino]-6-(tetrahydropyran-3-yloxy)-7-methoxy-quinazoline
4-[(3-chloro-4-fluoro-phenyl)amino]-6-(1-methyl-piperidin-4-yloxy)-7-methoxy-quinazoline
4-[(3-chloro-4-fluoro-phenyl)amino]-6-{1-[(morpholin-4-yl)carbonyl]-piperidin-4-yl-oxy}-7-methoxy-quinazoline
4-[(3-chloro-4-fluoro-phenyl)amino]-6-{1-[(methoxymethyl)carbonyl]-piperidin-4-yl-oxy}-7-methoxy-quinazoline
4-[(3-chloro-4-fluoro-phenyl)amino]-6-(piperidin-3-yloxy)-7-methoxy-quinazoline
4-[(3-chloro-4-fluoro-phenyl)amino]-6-[1-(2-acetylamino-ethyl)-piperidin-4-yloxy]-7-methoxy-quinazoline
4-[(3-chloro-4-fluoro-phenyl)amino]-6-(tetrahydropyran-4-yloxy)-7-ethoxy-quinazoline
4-[(3-chloro-4-fluoro-phenyl)amino]-6-((S)-tetrahydrofuran-3-yloxy)-7-hydroxy-quinazoline
4-[(3-chloro-4-fluoro-phenyl)amino]-6-(tetrahydropyran-4-yloxy)-7-(2-methoxy-ethoxy)-quinazoline
4-[(3-chloro-4-fluoro-phenyl)amino]-6-{trans-4-[(dimethylamino)sulphonylamino]-cyclohexan-1-yloxy}-7-methoxy-quinazoline
4-[(3-chloro-4-fluoro-phenyl)amino]-6-{trans-4-[(morpholin-4-yl)carbonylamino]-cyclohexan-1-yloxy}-7-methoxy-quinazoline
4-[(3-chloro-4-fluoro-phenyl)amino]-6-{trans-4-[(morpholin-4-yl)sulphonylamino]-cyclohexan-1-yloxy}-7-methoxy-quinazoline
4-[(3-chloro-4-fluoro-phenyl)amino]-6-(tetrahydropyran-4-yloxy)-7-(2-acetylamino-ethoxy)-quinazoline
4-[(3-chloro-4-fluoro-phenyl)amino]-6-(tetrahydropyran-4-yloxy)-7-(2-methanesulphonylamino-ethoxy)-quinazoline
4-[(3-chloro-4-fluoro-phenyl)amino]-6-{1-[(piperidin-1-yl)carbonyl]-piperidin-4-yloxy}-7-methoxy-quinazoline
4-[(3-chloro-4-fluoro-phenyl)amino]-6-(1-aminocarbonylmethyl-piperidin-4-yloxy)-7-methoxy-quinazoline
4-[(3-chloro-4-fluoro-phenyl)amino]-6-(cis-4-{N—[(tetrahydropyran-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline
4-[(3-chloro-4-fluoro-phenyl)amino]-6-(trans-4-ethansulphonylamino-cyclohexan-1-yloxy)-7-methoxy-quinazoline
4-[3-chloro-4-fluoro-phenyl)amino]-6-(1-methanesulphonyl-piperidin-4-yloxy)-7-ethoxy-quinazoline
4-[3-chloro-4-fluoro-phenyl)amino]-6-(1-methanesulphonyl-piperidin-4-yloxy)-7-(2-methoxy-ethoxy)-quinazoline
4-[(3-chloro-4-fluoro-phenyl)amino]-6-[1-(2-methoxy-acetyl)-piperidin-4-yloxy]-7-(2-methoxy-ethoxy)-quinazoline
4-[(3-chloro-4-fluoro-phenyl)amino]-6-(cis-4-acetylamino-cyclohexan-1-yloxy)-7-methoxy-quinazoline
4-[(3-ethynyl-phenyl)amino]-6-[1-(tert.-butyloxycarbonyl)-piperidin-4-yloxy]-7-methoxy-quinazoline
4-[(3-ethynyl-phenyl)amino]-6-(tetrahydropyran-4-yloxy]-7-methoxy-quinazoline
4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline
4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline
4-[(3-chloro-4-fluoro-phenyl)amino]-6-{cis-4-[(morpholin-4-yl)carbonylamino]-cyclohexan-1-yloxy}-7-methoxy-quinazoline
4-[(3-chloro-4-fluoro-phenyl)amino]-6-{1-[2-(2-oxopyrrolidin-1-yl)ethyl]-piperidin-4-yloxy}-7-methoxy-quinazoline
4-[(3-chloro-4-fluoro-phenyl)amino]-6-{1-[(morpholin-4-yl)carbonyl]-piperidin-4-yloxy}-7-(2-methoxy-ethoxy)-quinazoline
4-[(3-ethynyl-phenyl)amino]-6-(1-acetyl-piperidin-4-yloxy)-7-methoxy-quinazoline
4-[(3-ethynyl-phenyl)amino]-6-(1-methyl-piperidin-4-yloxy)-7-methoxy-quinazoline
4-[(3-ethynyl-phenyl)amino]-6-(1-methanesulphonyl-piperidin-4-yloxy)-7-methoxy-quinazoline
4-[(3-chloro-4-fluoro-phenyl)amino]-6-(1-methyl-piperidin-4-yloxy)-7(2-methoxy-ethoxy)-quinazoline
4-[(3-chloro-4-fluoro-phenyl)amino]-6-(1-isopropyloxycarbonyl-piperidin-4-yloxy)-7-methoxy-quinazoline
4-[(3-chloro-4-fluoro-phenyl)amino]-6-(cis-4-methylamino-cyclohexan-1-yloxy)-7-methoxy-quinazoline
4-[(3-chloro-4-fluoro-phenyl)amino]-6-{cis-4-[N-(2-methoxy-acetyl)-N-methyl-amino]-cyclohexan-1-yloxy}-7-methoxy-quinazoline
4-[(3-ethynyl-phenyl)amino]-6-(piperidin-4-yloxy)-7-methoxy-quinazoline
4-[(3-ethynyl-phenyl)amino]-6-[1-(2-methoxy-acetyl)-piperidin-4-yloxy]-7-methoxy-quinazoline
4-[(3-ethynyl-phenyl)amino]-6-{1-[(morpholin-4-yl)carbonyl]-piperidin-4-yloxy}-7-methoxy-quinazoline
4-[(3-chloro-4-fluoro-phenyl)amino]-6-{1-[(cis-2,6-dimethyl-morpholin-4-yl)carbonyl]-piperidin-4-yloxy}-7-methoxy-quinazoline
4-[(3-chloro-4-fluoro-phenyl)amino]-6-{1-[(2-methyl-morpholin-4-yl)carbonyl]-piperidin-4-yloxy}-7-methoxy-quinazoline
4-[(3-chloro-4-fluoro-phenyl)amino]-6-{1-[(S,S)-(2-oxa-5-aza-bicyclo[2,2,1]hept-5-yl)carbonyl}-piperidin-4-yloxy]-7-methoxy-quinazoline
4-[(3-chloro-4-fluoro-phenyl)amino]-6-{1-[(N-methyl-N-2-methoxyethyl-amino)carbonyl]-piperidin-4-yloxy}-7-methoxy-quinazoline
4-[(3-chloro-4-fluoro-phenyl)amino]-6-(1-ethyl-piperidin-4-yloxy)-7-methoxy-quinazoline
4-[(3-chloro-4-fluoro-phenyl)amino]-6-{1-[(2-methoxyethyl)carbonyl]-piperidin-4-yloxy}-7-methoxy-quinazoline
4-[(3-chloro-4-fluoro-phenyl)amino]-6-{1-[(3-methoxypropyl-amino)-carbonyl]-piperidin-4-yloxy}-7-methoxy-quinazoline
4-[(3-chloro-4-fluoro-phenyl)amino]-6-[cis-4-(N-methanesulphonyl-N-methyl-amino)-cyclohexan-1-yloxy]-7-methoxy-quinazoline
4-[(3-chloro-4-fluoro-phenyl)amino]-6-[cis-4-(N-acetyl-N-methyl-amino)-cyclohexan-1-yloxy]-7-methoxy-quinazoline
4-[3-chloro-4-fluoro-phenyl)amino]-6-(trans-4-methylamino-cyclohexan-1-yloxy)-7-methoxy-quinazoline
4-[3-chloro-4-fluoro-phenyl)amino]-6-[trans-4-(N-methanesulphonyl-N-methyl-amino)-cyclohexan-1-yloxy]-7-methoxy-quinazoline
4-[3-chloro-4-fluoro-phenyl)amino]-6-(trans-4-dimethylamino-cyclohexan-1-yloxy)-7-methoxy-quinazoline
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline
4-[(3-chloro-4-fluoro-phenyl)amino]-6-[2-(2.2-dimethyl-6-oxo-morpholin-4-yl)-ethoxy]-7-[(S)-(tetrahydrofuran-2-yl)methoxy]-quinazoline
4-[3-chloro-4-fluoro-phenyl)amino]-6-(1-methanesulphonyl-piperidin-4-yloxy)-7-methoxy-quinazoline
4-[3-chloro-4-fluoro-phenyl)amino]-6-(1-cyano-piperidin-4-yloxy)-7-methoxy-quinazoline
   optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptin, cabergoline, alpha-dihydroergocryptine, lisuride, pergolide, pramipexol, roxindol, ropinirol, talipexol, tergurid and viozan, optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorophenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The PAF-antagonists used are preferably compounds selected from among
4-(2-chlorophenyl)-9-methyl-2-[3(4-morpholinyl)-3-propanon-1-yl]-6H-thieno-[3,2-f]-[1,2,4]triazolo[4,3-a][1,4]diazepine
6-(2-chlorophenyl)-8,9-dihydro-1-methyl-8-[(4-morpholinyl)carbonyl]-4H,7H-cyclo-penta-[4,5]thieno-[3,2-f][1,2,4]triazolo[4,3-a][1,4]diazepine,
   optionally in the form of the racemates, enantiomers, diastereomers thereof and optionally in the form of the pharmacologically acceptable acid addition salts, solvates or hydrates thereof. According to the invention the preferred acid addition salts of the betamimetics are selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

### Formulations

Suitable formulations for administering the compounds of formula 1 include for example tablets, capsules, suppositories, solutions, powders etc. The content of the pharmaceutically active compound(s) should be in the range from 0.05 to 90 wt.-%, preferably 0.1 to 50 wt.-% of the composition as a whole. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates or stabilisers such as alkali metal salts of ethylenediaminetetraacetic acid, optionally using emulsifiers and/or dispersants, while if water is used as diluent, for example, organic solvents may optionally be used as solubilisers or dissolving aids, and the solutions may be transferred into injection vials or ampoules or infusion bottles.

Capsules containing the compounds of formula 1 according to the invention may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, preferably potato starch, gelatine and the like. Lubricants such as magnesium stearate, sodium laurylsulphate and talc may also be used to produce the tablets. In the case of aqueous suspensions the active substances may be combined with various flavour enhancers or colourings in addition to the above-mentioned excipients.

In the preferred use of the compounds of formula 1 for the treatment of respiratory complaints it is particularly preferred according to the invention to use preparations or pharmaceutical formulations which can be administered by inhalation. Inhalable preparations include inhalable powders, propellant-containing metered-dose aerosols or propellant-free inhalable solutions. Within the scope of the present invention, the term propellant-free inhalable solutions also includes concentrates or sterile ready-to-use inhalable solutions.

The compounds of formula 1 which are particularly preferably used in crystalline form according to the invention are preferably used to prepare powders for inhalation. The inhalable powders which may be used according to the invention may contain the crystalline compounds of formula 1 either on their own or in admixture with suitable physiologically acceptable excipients.

If the active substances are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose), oligo- and polysaccharides (e.g. dextrans), polyalcohols (e.g. sorbitol, mannitol, xylitol), salts (e.g. sodium chloride, calcium carbonate) or mixtures of these excipients. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. In some cases it may seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance, preferably with an average particle size of 0.5 to 10 μm, more preferably from 1 to 5 μm, is added to the excipient mixture. Processes for producing the inhalable powders according to the invention by grinding and micronising and finally mixing the ingredients together are known from the prior art.

The inhalable powders according to the invention may be administered using inhalers known from the prior art.

The inhalation aerosols containing propellant gas according to the invention may contain dissolved in the propellant gas or in dispersed form. The propellant gases which may be used to prepare the inhalation aerosols are known from the prior art. Suitable propellant gases are selected from among hydrocarbons such as n-propane, n-butane or isobutane and halohydrocarbons such as fluorinated derivatives of methane, ethane, propane, butane, cyclopropane or cyclobutane. The above-mentioned propellant gases may be used on their own or in admixture. Particularly preferred propellant gases are halogenated alkane derivatives selected from TG134a and TG227 and mixtures thereof.

The propellant-driven inhalation aerosols may also contain other ingredients such as co-solvents, stabilisers, surfactants, antioxidants, lubricants and pH adjusters. All these ingredients are known in the art.

The propellant-driven inhalation aerosols mentioned above may be administered using inhalers known in the art (MDIs=metered dose inhalers).

The dosage of the compounds according to the invention is naturally highly dependent on the method of administration and the complaint which is being treated. When administered by inhalation the compounds of the formula are characterised by a high potency even at doses in the ng range. The compounds of the formula may also be used effectively above the ng range. The dosage may then be in the milligram range, for example.

In another aspect the present invention relates to the above-mentioned pharmaceutical formulations as such, which are characterised in that they contain a compound of formula I, particularly preferably the above-mentioned pharmaceutical formulations administered by inhalation.

The following examples of formulations illustrate the present invention without restricting its scope:

### Examples of Pharmaceutical Formulations

| A) | Tablets | per tablet |
|---|---|---|
| | active substance | 100 mg |
| | lactose | 140 mg |
| | corn starch | 240 mg |
| | polyvinylpyrrolidone | 15 mg |
| | magnesium stearate | 5 mg |
| | | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet | |
|---|---|---|---|
| | active substance | 80 | mg |
| | corn starch | 190 | mg |
| | lactose | 55 | mg |
| | microcrystalline cellulose | 35 | mg |
| | polyvinylpyrrolidone | 15 | mg |
| | sodium-carboxymethyl starch | 23 | mg |
| | magnesium stearate | 2 | mg |
| | | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium carboxymethyl starch and the magnesium stearate are added and mixed in and the mixture is compressed to form tablets of a suitable size.

| C) | Coated tablets | per coated tablet | |
|---|---|---|---|
| | Active substance | 5 | mg |
| | Corn starch | 41.5 | mg |
| | Lactose | 30 | mg |
| | Polyvinylpyrrolidone | 3 | mg |
| | Magnesium stearate | 0.5 | mg |
| | | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| D) | Capsules | per capsule | |
|---|---|---|---|
| | Active substance | 50 | mg |
| | Corn starch | 268.5 | mg |
| | Magnesium stearate | 1.5 | mg |
| | | 320 | mg |

The substance and corn starch are mixed and moistened with water. The moist mass is screened and dried. The dry granules are screened and mixed with magnesium stearate. The finished mixture is packed into size 1 hard gelatine capsules.

| E) | Ampoule solution | |
|---|---|---|
| | active substance | 50 mg |
| | sodium chloride | 50 mg |
| | water for inj. | 5 ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

| | |
|---|---|
| F) | Suppositories |

| | |
|---|---|
| Active substance | 50 mg |
| Solid fat | 1650 mg |
| | 1700 mg |

The hard fat is melted. At 40° C. the ground active substance is homogeneously dispersed therein. It is cooled to 38° C. and poured into slightly chilled suppository moulds.

| G) | Oral suspension | | |
|---|---|---|---|
| | active substance | 50 | mg |
| | hydroxyethylcellulose | 50 | mg |
| | sorbic acid | 5 | mg |
| | sorbitol (70%) | 600 | mg |
| | glycerol | 200 | mg |
| | flavouring | 15 | mg |
| | water ad | 5 | ml |

Distilled water is heated to 70° C. Hydroxyethyl-cellulose is dissolved therein with stirring. After the addition of sorbitol solution and glycerol the preparation is cooled to ambient temperature. At ambient temperature the sorbic acid, flavouring and substance are added. The suspension is evacuated with stirring to eliminate any air.

1. Enantiomerically pure compounds of formula 1 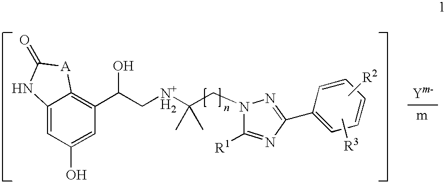
wherein
n denotes 1, 2, 3 or 4;
A denotes a double-bonded group selected from among O, S, CR⁴R⁵, CR⁴R⁵—O, CR⁴R⁵—NR⁶, CH═CH or CH₂—CH₂;
R¹ denotes C₁₋₆-alkyl;
R² and R³ which may be identical or different represent H, C₁₋₆-alkyl, C₂₋₆-alkenyl, C₂₋₆-alkynyl, C₃₋₆-cycloalkyl, C₁₋₆-haloalkyl, O—C₁₋₆-haloalkyl, halogen, OH, CN, NO₂, O—C₁₋₆-alkyl, C₂₋₆-alkyl-OH, NH₂, NH—C₁₋₆-alkyl, N(C₁₋₆-alkyl)₂, NHCO—C₁₋₆-alkyl, NHSO₂—C₁₋₆-alkyl, S—C₁₋₆-alkyl, SO—C₁₋₆-alkyl, SO₂—C₁₋₆-alkyl, SO₂NH₂, SO₂NH—C₁₋₆-alkyl, SO₂N(C₁₋₆-alkyl)₂, CONH₂, CONH—C₁₋₆-alkyl, CON(C₁₋₆-alkyl)₂, CO—C₁₋₆-alkyl, COOH or COO—C₁₋₄-alkyl, or
R² and R³ together denote a 2-bonded group selected from O—CR⁴R⁵—O, O—CR⁴R⁵—NR⁶ or CH═CH—CH═CH;
R⁴ denotes H or C₁₋₆-alkyl;
R⁵ denotes H or C₁₋₆-alkyl;
R⁶ denotes H or C₁₋₆-alkyl;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, propanedisulphonate, benzoate and p-toluenesulphonate;
m denotes 1 or 2;
optionally in the form of the tautomers, mixtures of the tautomers, hydrates or solvates thereof.

2. Enantiomerically pure compounds of formula 1 according to claim 1, wherein
n denotes 1, 2 or 3, preferably 2;
A denotes a double-bonded group selected from among CR⁴R⁵—O, CH═CH or CH₂—CH₂, preferably CR⁴R⁵—O;
R¹ denotes C₁₋₄-alkyl;
R² and R³ which may be identical or different represent H, C₁₋₄-alkyl, C₂₋₄-alkenyl, C₂₋₄-alkynyl, C₃₋₆-cycloalkyl, C₁₋₄-haloalkyl, O—C₁₋₄-haloalkyl, halogen, OH, CN, NO₂, C₂₋₄-alkyl-OH, O—C₁₋₄-alkyl, COOH or COO—C₁₋₄-alkyl, or
R² and R³ together denote a 2-bonded group selected from O—CR⁴R⁵—O, O—CR⁴R⁵—NR⁶ or CH═CH—CH═CH;
R⁴ denotes H or C₁₋₄-alkyl;
R⁵ denotes H or C₁₋₄-alkyl;
R⁶ denotes H or C₁₋₄-alkyl;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate;
m denotes 1 or 2;
optionally in the form of the tautomers, mixtures of the tautomers, hydrates or solvates thereof.

3. Enantiomerically pure compounds of formula 1 according to claim 1, wherein
A denotes a double-bonded group selected from among CR⁴R⁵—O, CH═CH or CH₂—CH₂, preferably CR⁴R⁵—O wherein
R⁴ denotes H, methyl, ethyl, preferably H or methyl, particularly preferably H;
R⁵ denotes H, methyl, ethyl, preferably H or methyl, particularly preferably H;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate,
m denotes 1 or 2
optionally in the form of the tautomers, mixtures of the tautomers, hydrates or solvates thereof.

4. Enantiomerically pure compounds of formula 1 according to claim 1, wherein
R² denotes H, methyl, ethyl, propyl, vinyl, allyl, propargyl, cyclopropyl, cyclopentyl, cyclohexyl, CH₂Cl, CHCl₂, CCl₃, CH₂F, CHF₂, CF₃, CH₂—CH₂Cl, CH₂—CHCl₂, CH₂—CCl₃, CH₂—CH₂F, CH₂—CHF₂, CH₂—CF₃, CH₂—CH₂OH, fluorine, chlorine, bromine, OH, CN, NO₂, methoxy, ethoxy, propoxy, COOH, COO-methyl, COO-ethyl, COO-propyl or COO-butyl;
R³ methyl, ethyl, propyl, vinyl, allyl, propargyl, cyclopropyl, cyclopentyl, cyclohexyl, CH₂Cl, CHCl₂, CCl₃, CH₂F, CHF₂, CF₃, CH₂—CH₂Cl, CH₂—CHCl₂, CH₂—CCl₃, CH₂—CH₂F, CH₂—CHF₂, CH₂—CF₃, CH₂—CH₂OH, fluorine, chlorine, bromine, OH, CN, NO₂, methoxy, ethoxy, propoxy, COOH, COO-methyl, COO-ethyl, COO-propyl or COO-butyl, or
R² and R³ together denote a 2-bonded group selected from O—CR⁴R⁵—O, O—CR⁴R⁵—NR⁶ or CH═CH—CH═CH;
R⁴ denotes H, methyl, ethyl, preferably H or methyl, particularly preferably H;
R⁵ denotes H, methyl, ethyl, preferably H or methyl, particularly preferably H;
R⁶ denotes H, methyl, ethyl, preferably H or methyl, particularly preferably H;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate,
m denotes 1 or 2,
optionally in the form of the tautomers, mixtures of the tautomers, hydrates or solvates thereof.

5. Enantiomerically pure compounds of formula 1 according to claim 1, wherein
R² denotes H, methyl, ethyl, CF₃, CH₂—CF₃, fluorine, chlorine, OH, methoxy, ethoxy, COOH or COO-methyl;
R³ denotes methyl, ethyl, propyl, vinyl, allyl, cyclopropyl, cyclopentyl, cyclohexyl, CH₂F, CHF₂, CF₃, CH₂—CH₂F, CH₂—CHF₂, CH₂—CF₃, CH₂—CH₂OH, fluorine, chlorine, OH, CN, methoxy, ethoxy, COOH, COO-methyl, COO-ethyl or COO-butyl, or
R² and R³ together denote a 2-bonded group selected from O—CH₂—O, O—CMe₂-O or CH═CH—CH═CH;
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate,
m denotes 1 or 2,
optionally in the form of the tautomers, mixtures of the tautomers, hydrates or solvates thereof.

6. Enantiomerically pure compounds of formula
1.2: 8-{2-[1,1-dimethyl-3-(5-methyl-3-p-tolyl-[1,2,4]triazol-1yl)-propylamino]-1-hydroxy-ethyl}-6-hydroxy-4H-benzo[1,4]oxazin-3-one *H(Y^{m-}/m);
1.3: 8-(2-{1,1-dimethyl-3-[5-methyl-3-(4-trifluoromethyl-phenyl)-[1,2,4]triazol-1-yl]-propylamino}-1-hydroxy-ethyl)-6-hydroxy-4H-benzo[1,4]oxazin-3-one*H(Y^{m-}/m);
1.6: 8-(2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.7: 8-(2-{3-[5-ethyl-3-(4-methoxy-phenyl)-[1,2,4]triazol-1-yl]-1,1-dimethyl-propylamino}-1-hydroxy-ethyl)-6-hydroxy-4H-benzo[1,4]oxazin-3-one*H(Y^{m-}/m);
1.9: 8-{2-[3-(3-benzo[1,3]dioxol-5-yl-5-methyl-[1,2,4]triazol-1-yl)-1,1-dimethyl-propylamino]-1-hydroxy-ethyl}-6-hydroxy-4H-benzo[1,4]oxazin-3-one*H(Y^{m-}/m); according to claim 1, wherein
Y^{m-} denotes an anion with m negative charges, preferably an anion with m negative charges selected from among chloride, bromide, iodide, sulphate, phosphate, methanesulphonate, nitrate, maleinate, acetate, benzoate, citrate, malate, lactate, salicylate, trifluoroacetate, fumarate, tartrate, oxalate, succinate, ethanedisulphonate, benzoate and p-toluenesulphonate,
m denotes 1 or 2
optionally in the form of the tautomers, mixtures of the tautomers, hydrates or solvates thereof.

7. Enantiomerically pure compounds of general formula 1 according to claim 1, characterised in that they are present in crystalline form, optionally in the form of the crystalline tautomers, crystalline hydrates or crystalline solvates thereof.

8. Compounds according to claim 1 of formula:
8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one benzoate;
8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one acetate;
8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one L-lactate;
8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one maleate;
8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one malate;
8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one, hydrobromide dihydrate and
8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one hydrochloride dihydrate.

9. Crystalline 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one benzoate, characterised in that it has an endothermic peak at 125° C.

10. Crystalline compound according to claim 9, characterised in that it has X-ray reflections at d=10.62, 8.62, 5.34, 4.43 and 3.54 A.

11. Crystalline 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one acetate, characterised in that it has an endothermic peak at 160° C.

12. Crystalline compound according to claim 11, characterised in that it has X-ray reflections at d=6.34, 3.97, 3.94 and 3.42 A.

13. Crystalline 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one L-lactate, characterised in that it has an endothermic peak at 215° C.

14. Crystalline compound according to claim 14, characterised in that it has X-ray reflections at d=6.00, 5.54, 4.17 and 3.61 Å.

15. Crystalline 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one maleate, characterised in that it has an endothermic peak at 230° C.

16. Crystalline compound according to claim 15, characterised in that it has X-ray reflections at d=4.83, 4.65, 3.88 and 3.47 Å.

17. Crystalline 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one malate, characterised in that it has an endothermic peak at 200° C.

18. Crystalline compound according to claim 17, characterised in that it has X-ray reflections at d=6.63, 5.76 and 3.87 Å.

19. Crystalline 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one hydrobromide dihydrate, characterised in that it has an endothermic peak at 165° C.

20. Crystalline compound according to claim 19, characterised in that it has X-ray reflections at d=6.95, 4.80, 4.39 and 3.46 Å.

21. Crystalline 8-((R)-2-{3-[3-(4-chloro-phenyl)-5-methyl-[1,2,4]triazol-1-yl]-1,1-dimethyl-propylamino}-1-hydroxy-ethyl)-6-hydroxy-4H-benzo[1,4]oxazin-3-one hydrochloride dihydrate, characterised in that it has an endothermic peak at 170° C.

22. Crystalline compound according to claim 21, characterised in that it has X-ray reflections at d=4.94, 4.76, 4.27, 3.89 and 3.44 Å.

23. Enantiomerically pure compounds of formula 1 according to claim 1 as pharmaceutical compositions.

24. A method for the treatment of respiratory complaints comprising administering to a patient a pharmaceutical composition according to claim 1.

25. Pharmaceutical formulation, characterised in that it contains a compound of formula 1 according to claim 1.
